(19) United States
(12) Patent Application Publication
(10) Pub. No.: US 2023/0392140 A1
(43) Pub. Date: Dec. 7, 2023

(54) REVERSE TRANSCRIPTION OF POLYNUCLEOTIDES COMPRISING UNNATURAL NUCLEOTIDES

(71) Applicants: Synthorx, Inc., La Jolla, CA (US); The Scripps Research Institute, La Jolla, CA (US)

(72) Inventors: Floyd E. ROMESBERG, La Jolla, CA (US); Xiyu Dong, Shandong Province (CN); Anne Xiaozhou Zhou, La Jolla, CA (US)

(21) Appl. No.: 18/304,993

(22) Filed: Apr. 21, 2023

Related U.S. Application Data
(63) Continuation of application No. PCT/US2021/056334, filed on Oct. 22, 2021.
(60) Provisional application No. 63/104,785, filed on Oct. 23, 2020.

(51) Int. Cl. C12N 15/10; C12N 9/12

(52) U.S. Cl. CPC C12N 15/1048; C12N 9/1276; C12Y 207/07049; C12N 15/1096

(57) ABSTRACT

Disclosed herein are methods of reverse transcribing a polynucleotide comprising an unnatural ribonucleotide comprising reverse transcribing the polynucleotide with a reverse transcriptase in the presence of an unnatural dNTP comprising an unnatural nucleobase, wherein the reverse transcriptase polymerizes cDNA into which the unnatural NTP is incorporated. In some embodiments, the polynucleotide is present at a concentration less than or equal to about 500 nM and/or the polynucleotide is a tRNA, mRNA, RNA aptamer, or a member of a plurality of RNA aptamer candidates.

### CROSS-REFERENCE TO RELATED APPLICATIONS

This application is a continuation of International Application No. PCT/US2021/056334, filed Oct. 22, 2021, which claims the benefit of U.S. Provisional Patent Application No. 63/104,785, filed on Oct. 23, 2020, all of which are herein incorporated by reference in their entireties for all purposes.

### STATEMENT AS TO FEDERALLY SPONSORED RESEARCH

This invention was made with government support under Grant No. GM118178 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on August 14, 2023, is named “2023-08-14-01183-5012-00US-ST26.xml” and is 42,521 bytes in size.

### INTRODUCTION AND SUMMARY

Upon its discovery, the 61 sense codon/20 amino acid genetic code was considered invariant, conserved across all living organisms. However, intensive characterization revealed unexpected plasticity with altered codon assignments and even, in rare cases, expansion to include the non-canonical amino acids (ncAAs) selenocysteine or pyrrolysine. (Yuan, J., et al. FEBS Lett. 2010, 584, 342-349; Hao, B., et al. Science 2002, 296, 1462-1466; Kryukov, G. V., et al. Science 2003, 300, 1439-1443.) All of these alterations result from reassignments of natural codons, and a similar strategy forms the basis of significant efforts to expand the code to include ncAAs of interest, by utilizing stop codons and orthogonal pairs of recoded suppressor tRNAs/amino acyl tRNA synthetases (aaRS). (Xiao, H. et al. Cold Spring Harb. Perspect. Biol. 2016, 8; Wang, L. et al. Annu. Rev. Biophys. Biomol. Struct. 2006, 35, 225-249.) An alternative to these reassignment strategies is to focus on the creation of new codons via the development of unnatural base pairs (UBPs). (Malyshev, D. A. et al., Nature 2014, 509, 385-388; Zhang, Y., et al. Nature 2017, 551, 644-647.) Most notably, several UBPs, including the (d)NaM-(d)TPT3 UBP (FIG. 1) have been used to create E. coli-based semi-synthetic organisms (SSOs) that retain UBPs in their DNA, transcribe them into mRNA and tRNA, and when provided with an aaRS that selectively aminoacylates the unnatural anticodon-bearing tRNA with a ncAA, use them to translate proteins containing the ncAA.

While the (d)NaM-(d)TPT3 UBP is able to produce unnatural proteins, the efficiency with which the ncAA is incorporated depends on its sequence context, such that some codons are more efficient than others. Examining sequence context, a number of codons have been identified that are efficiently replicated as DNA and then efficiently transcribed into RNA and decoded at the ribosome. (Fischer, E. C., et al. Nat. Chem. Biol. 2020, 16, 570-576.) As assays for the retention of the UBP in the DNA of the SSO are available, the reduced fidelity of several of the less efficient codons is known to result from either poor transcription or poor translation. However, the lack of an assay to measure transcription fidelity has prevented the identification of the specific step that compromises fidelity. In addition, while it is clear that different DNA polymerases, T7 RNA polymerase, and E. coli ribosomes are able to productively recognize the UBP, the ability of reverse transcriptases, which mediate the only other common DNA/RNA transaction, has not been thoroughly explored, and the only available data suggests that they might not productively recognize the UBP. (Eggert et al., Towards Reverse Transcription with an Expanded Genetic Alphabet. Chembiochem 2019, 20, 1642-1645.) Accordingly, there is a need for methods for reverse transcribing polynucleotides comprising an unnatural nucleotide, and for methods that can determine the fidelity of transcription and reverse transcription such that the fidelity of SSO ncAA incorporation into a protein can be understood in terms of the relative contribution of transcription and translation.

Additionally, RNA oligonucleotides can function as aptamers that recognize a specific target, e.g., for purposes of inhibiting or detecting the target. However, the screening and selection of RNA aptamers from oligonucleotide libraries (large mixtures of oligonucleotides with different sequences of nucleotides) generally involves a reverse transcription step to convert the RNA into cDNA. Accordingly, to develop RNA aptamers comprising unnatural nucleotides, there is also a need for methods of reverse transcribing RNA comprising unnatural nucleotides.

Accordingly, the following embodiments are provided. Embodiment 1 is a method of reverse transcribing a polynucleotide comprising an unnatural ribonucleotide, comprising reverse transcribing the polynucleotide with a reverse transcriptase in the presence of an unnatural dNTP comprising an unnatural nucleobase,

wherein the reverse transcriptase polymerizes a cDNA into which the unnatural dNTP is incorporated as an unnatural nucleotide.

Embodiment 2 is the method of embodiment 1, wherein:
the polynucleotide is present at a concentration less than or equal to about 500 nM.

Embodiment 2.1 is the method of any one of the preceding embodiments, wherein the reverse transcriptase is SuperScript III.

Embodiment 2.2 is the method of any one of the preceding embodiments, wherein the unnatural dNTP is not dTPT3TP.

Embodiment 2.3 is the method of any one of the preceding embodiments, wherein the method further comprises measuring the amount of the unnatural nucleotide in the cDNA using a binding partner that recognizes the unnatural nucleotide.

Embodiment 2.4 is the method of any one of the preceding embodiments, wherein the reverse transcriptase produces full length cDNA and at least 25% of the full length cDNA comprises the unnatural nucleotide.

Embodiment 2.5 is the method of any one of the preceding embodiments, wherein the polynucleotide is a tRNA, mRNA, RNA aptamer, or a member of a plurality of RNA aptamer candidates.

Embodiment 3 is the method of any one of the preceding embodiments, wherein the polynucleotide is an RNA, optionally wherein the RNA is an mRNA or tRNA.

Embodiment 4 is the method of any one of embodiments 1-3, further comprising measuring the amount of the unnatural nucleotide in the cDNA.

Embodiment 5 is a method of measuring incorporation of an unnatural nucleotide, comprising:
a. transcribing a polynucleotide comprising an unnatural deoxyribonucleotide with an RNA polymerase in the presence of an unnatural NTP comprising a first unnatural nucleobase to produce an RNA comprising a first unnatural nucleotide;
b. reverse transcribing the RNA with a reverse transcriptase in the presence of an unnatural dNTP comprising a second unnatural nucleobase,
   wherein the reverse transcriptase polymerizes a cDNA into which the unnatural NTP is incorporated as a second unnatural nucleotide; and
c. measuring the amount of the second unnatural nucleotide in the cDNA.

Embodiment 5.1 is the method of embodiment 5, which is a method of measuring combined fidelity of transcription and reverse transcription.

Embodiment 5.2 is the method of embodiment 5, which is a method of measuring retention of an unnatural nucleotide during transcription and reverse transcription.

Embodiment 6 is the method of any one of embodiments 5-5.2, wherein the transcribing step is in vivo.

Embodiment 7 is the method of the immediately preceding embodiment, wherein the transcribing step is in a prokaryote or bacterium.

Embodiment 8 is the method of the immediately preceding embodiment, wherein the transcribing step is in E. coli.

Embodiment 9 is the method of embodiment 5, wherein the transcribing step is in vitro.

Embodiment 10 is the method of any one of embodiments 5-9, wherein the amount of the second unnatural nucleotide in the cDNA molecule is measured relative to the amount of the unnatural deoxyribonucleotide in the polynucleotide before transcription.

Embodiment 11 is the method of any one of embodiments 5-10, wherein the measuring comprises:
a. performing a biotin shift assay on the polynucleotide before transcription to determine the proportion of the polynucleotide before transcription that contains the unnatural nucleotide; and
b. performing a biotin shift assay on the cDNA to determine the proportion of the cDNA that contains containing the unnatural nucleotide.

Embodiment 12 is the method of any one of embodiments 4-10, wherein the amount of the unnatural nucleotide or the second unnatural nucleotide in the cDNA is measured using a binding partner that binds an unnatural nucleobase.

Embodiment 13 is the method of any one of embodiments 4-10, wherein measuring the amount of the unnatural nucleotide or the second unnatural nucleotide in the cDNA comprises a gel shift assay or biotin shift assay.

Embodiment 14 is the method of the immediately preceding embodiment, wherein the biotin shift assay comprises:
a. amplifying the cDNA in the presence of an unnatural dNTP comprising a biotinylated nucleobase that pairs with the unnatural nucleotide in the cDNA;
b. separating DNA amplification products comprising the biotinylated nucleotide from DNA amplification products not comprising the biotinylated nucleotide; and
c. measuring the amount of DNA amplification products comprising the biotinylated nucleotide and DNA amplification products not comprising the biotinylated nucleotide, or a ratio of DNA amplification products comprising the biotinylated nucleotide to DNA amplification products not comprising the biotinylated nucleotide, or the proportion of cDNA that contains the unnatural nucleotide.

Embodiment 15 is the method of the immediately preceding embodiment, wherein separating DNA amplification products comprising the biotinylated nucleotide from DNA amplification products not comprising the biotinylated nucleobase comprises gel electrophoresis, optionally wherein the gel electrophoreses is polyacrylamide gel electrophoresis.

Embodiment 16 is the method of any one of embodiments 14-15, wherein separating DNA amplification products comprising the biotinylated nucleotide from DNA amplification products not comprising the biotinylated nucleotide comprises incubating the amplification products with streptavidin.

Embodiment 17 is the method of any one of the preceding embodiments, wherein the RNA or polynucleotide is present during reverse transcription at a concentration less than or equal to about 1 μM.

Embodiment 18 is the method of any one of the preceding embodiments, wherein the RNA or polynucleotide is present during reverse transcription at a concentration in the range of about 1-10 nM, about 10-20 nM, about 20-30 nM, about 30-40 nM, about 40-50 nM, about 50-nM, about 75-100 nM, about 100-150 nM, about 150-200 nM, about 200-300 nM, about 300-400 nM, or about 400-500 nM.

Embodiment 19 is the method of any one of the preceding embodiments, wherein the reverse transcriptase produces full length cDNA and wherein at least 25% of the full length cDNA comprises the unnatural nucleotide.

Embodiment 20 is the method of the immediately preceding embodiment, wherein at least 50%, 75%, 80%, 85%, 90%, 95%, 97%, 98%, or 99% of the non-truncated cDNA comprises the unnatural nucleotide.

Embodiment 21 is the method of any one of the preceding embodiments, wherein the RNA or polynucleotide comprising the unnatural ribonucleotide is an mRNA.

Embodiment 22 is the method of embodiment 20, wherein the unnatural ribonucleotide (X or Y) is located at the first position (X—N—N or Y—N—N) of a codon of the mRNA.

Embodiment 23 is the method of embodiment 20, wherein the unnatural ribonucleotide (X or Y) is located at the middle position (N—X—N or N—Y—N) of a codon of the mRNA.

Embodiment 24 is the method of embodiment 20, wherein the unnatural ribonucleotide (X or Y) is located at the last position (N—N—X or N—N—Y) of a codon of the mRNA.

Embodiment 25 is the method of any one of embodiments 51-25, wherein the codon containing the unnatural ribonucleotide in the mRNA is AXC, AYC, GXC, GYC, GXT, GYT, AXA, AXT, TXA, or TXT.

Embodiment 26 is the method of any one of embodiments 1-20, wherein the RNA or polynucleotide comprising the unnatural ribonucleotide is a tRNA.

Embodiment 27 is the method of embodiment 26, wherein the unnatural ribonucleotide (X or Y) is located at the first position (X—N—N or Y—N—N) of the anticodon of the tRNA.

Embodiment 28 is the method of embodiment 26, wherein the unnatural ribonucleotide (X or Y) is located at the middle position (N—X—N or N—Y—N) of the anticodon of the tRNA.

Embodiment 29 is the method of embodiment 26, wherein the unnatural ribonucleotide (X or Y) is located at the last position (N—N—X or N—N—Y) of the anticodon of the tRNA.

Embodiment 30 is the method of any one of embodiments 26-29, wherein the anticodon of the tRNA is GYT, GXT, GYC, GXC, CYA, CXA, AYC, or AXC. Embodiment 31 is the method of any one of embodiments 1-30, wherein the unnatural ribonucleotide is X, wherein X comprises

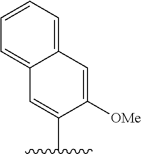 [SMILES: *c1cc2ccccc2cc1OC]

as the nucleobase of the unnatural ribonucleotide (NaM).

Embodiment 32 is the method of any one of embodiments 1-30, wherein the unnatural ribonucleotide is Y, wherein Y comprises

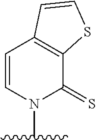 [SMILES: *n1ccc2ccsc2c1=S]

as the nucleobase of the unnatural ribonucleotide (TPT3).

Embodiment 33 is the method of any one of embodiments 1-20 or 31-32, wherein the RNA is an RNA aptamer.

Embodiment 34 is a method of screening RNA aptamer candidates comprising:
a. incubating a plurality of different RNA oligonucleotides with a target, wherein the RNA oligonucleotides comprise at least one unnatural nucleotide;
b. performing at least one round of selection for RNA oligonucleotides of the plurality that bind to the target;
c. isolating enriched RNA oligonucleotides that bind to the target, wherein the isolated enriched RNA oligonucleotides comprise RNA aptamers; and
d. reverse transcribing one or more of the RNA aptamers into cDNAs, wherein the cDNAs comprise an unnatural deoxyribonucleotide at the position complementary to the at least one unnatural nucleotide in the RNA aptamer, thereby providing a library of cDNA molecules corresponding to the RNA aptamers.

Embodiment 35 is the method of the immediately preceding embodiment, wherein the plurality of different RNA oligonucleotides comprise a randomized nucleotide region.

Embodiment 36 is the method of the immediately preceding embodiment, wherein the randomized nucleotide region comprises the at least one unnatural nucleotide.

Embodiment 37 is the method of any one of embodiments 34-36, wherein the RNA oligonucleotides comprise barcode sequences and/or primer binding sequences.

Embodiment 38 is the method of any one of embodiments 34-37, wherein the method further comprises sequencing the cDNA molecules.

Embodiment 39 is the method of any one of embodiments 34-38, wherein performing at least one round of selection comprises a wash step to remove unbound or weakly bound RNA oligonucleotides.

Embodiment 40 is the method of any one of embodiments 34-39, wherein the method further comprises mutating the sequence of the cDNA molecules to generate a plurality of additional sequences.

Embodiment 41 is the method of the immediately preceding embodiment, wherein the plurality of additional sequences is transcribed into RNA and subjected to at least one additional round of selection for RNA aptamers that bind to the target.

Embodiment 42 is the method of any one of embodiments 40-41, wherein mutating the sequence of the cDNA molecules comprises error-prone PCR.

Embodiment 43 is the method of any one of embodiments 34-42, wherein the method further comprises increasing selection pressure for binding to the target in an additional round of selection.

Embodiment 44 is the method of the immediately preceding embodiment, wherein increasing selection pressure comprises performing one or more washing steps at a higher salt concentration than in a previous round and/or including a binding competitor during the selection.

Embodiment 45 is the method of any one of embodiments 34-44, further comprising analyzing the RNA aptamers for their ability to bind the target.

Embodiment 46 is the method of the immediately preceding embodiment, wherein analyzing the RNA aptamers for their ability to bind the target comprises determining a K_{d}, kₒₙ, or k_{off}.

Embodiment 47 is the method of any one of embodiments 34-44, further comprising analyzing the RNA aptamers for their ability to agonize the target.

Embodiment 48 is the method of the immediately preceding embodiment, wherein analyzing the RNA aptamers for their ability to agonize the target comprises determining an EC₅₀ value.

Embodiment 49 is the method of any one of embodiments 34-44, further comprising analyzing the RNA aptamers for their ability to antagonize the target.

Embodiment 50 is The method of the immediately preceding embodiment, wherein analyzing the RNA aptamers for their ability to antagonize the target comprises determining a Kᵢ or IC₅₀ value.

Embodiment 51 is the method of any one of the preceding embodiments, wherein at least one unnatural nucleotide comprises:

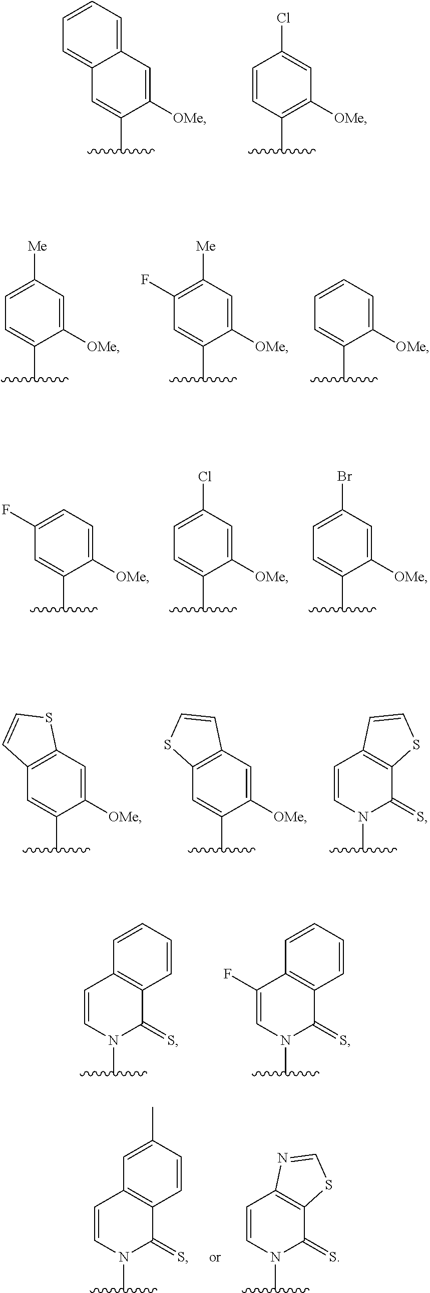

Embodiment 52 is the method of the immediately preceding embodiment, wherein at least one unnatural nucleotide in a polynucleotide that undergoes reverse transcription comprises:

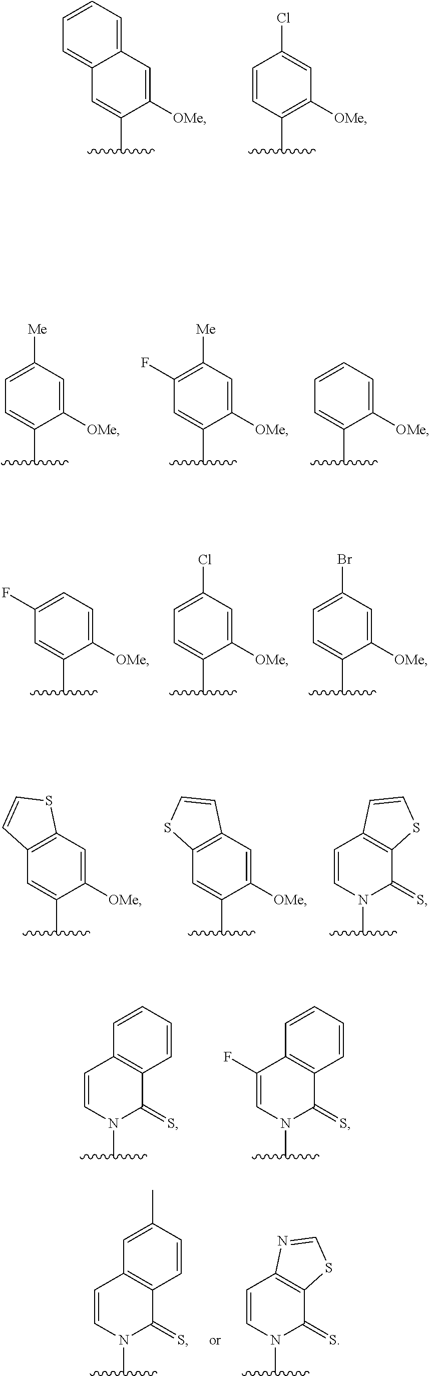

Embodiment 53 is the method of embodiment 51 or 52, wherein at least one unnatural nucleotide that is incorporated into cDNA comprises:

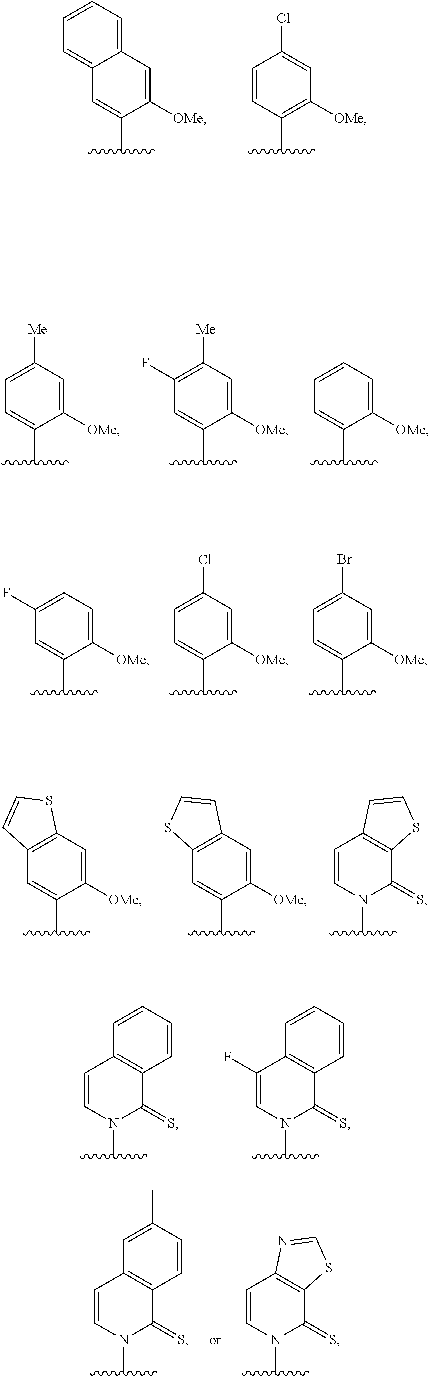

and optionally wherein the at least one unnatural nucleobase in the unnatural nucleotide is different from the at least one unnatural nucleobase in the polynucleotide that undergoes reverse transcription.

Embodiment 54 is the method of any one of embodiments 51-53, wherein the at least one unnatural nucleotidee comprises:

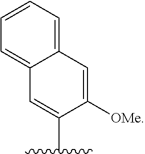 [SMILES: *c1cc2ccccc2cc1OC]

Embodiment 55 is the method of embodiments 51-53, wherein the at least one unnatural nucleotide comprises:

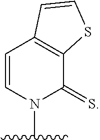 [SMILES: *n1ccc2ccsc2c1=S]

Embodiment 56 is the method of any one of the preceding embodiments, wherein the reverse transcriptase is Avian Myeloblastosis Virus (AMV) reverse transcriptase, Moloney Murine Leukemia Virus (MMLV) reverse transcriptase, Super Script II (SS II) reverse transcriptase, Super Script III (SS III) reverse transcriptase, Super Script IV (SS IV) reverse transcriptase, or Volcano 2G (V2G) reverse transcriptase.

Embodiment 57 is the method of any one of the preceding embodiments, wherein the reverse transcriptase is SuperScript III.

Embodiment 58 is the method of any one of the preceding embodiments, wherein the unnatural dNTP is not dTPT3TP.

Embodiment 59 is the method of any one of the preceding embodiments, wherein the reverse transcribing takes place in vitro.

### BRIEF DESCRIPTION OF THE DRAWINGS

Various aspects of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 shows unnatural base pairs between dNAM and dTPT3, and between NaM and TPT3.

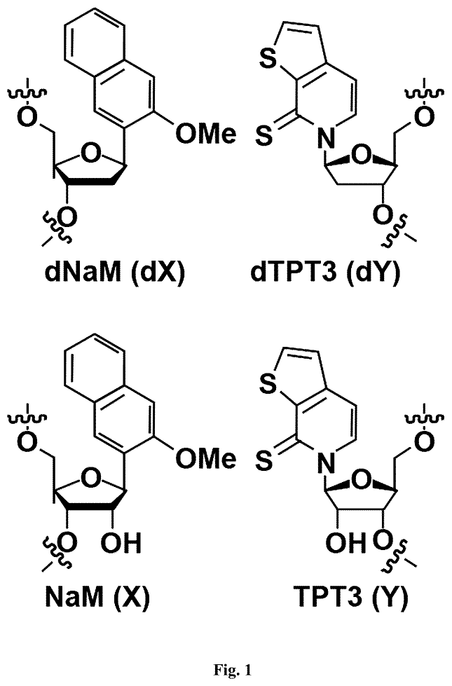

FIG. 2 shows a denaturing gel for cDNA detection and qualitative biotin shift of cDNA in different reverse transcription (RT) reaction conditions.

FIG. 3 shows full-length cDNA ratio as a function of RNA concentration in RT reactions using SuperScript III.

FIG. 4 shows a schematic of an exemplary transcription-reverse transcription (T-RT) process for measuring unnatural nucleotide retention.

FIGS. 5A-B show fidelity levels in T-RT retention assays for sequences comprising the indicated codons.

FIG. 6 shows images of denaturing gels for cDNA detection with different codons and anticodons.

FIGS. 7A-B show T-RT retention of mRNA from in vivo translation experiments for sequences comprising the indicated codons (with previously reported protein shift values shown below where available).

FIGS. 8A-B show dependency of mRNA transcription fidelity on NaMTP concentration of TPT3TP concentration, respectively, in an in vivo translation experiment.

### DETAILED DESCRIPTION

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

An “analog” of a chemical structure, as the term is used herein, refers to a chemical structure that preserves substantial similarity with the parent structure, although it may not be readily derived synthetically from the parent structure. In some embodiments, a nucleotide analog is an unnatural nucleotide. In some embodiments, a nucleoside analog is an unnatural nucleoside. A related chemical structure that is readily derived synthetically from a parent chemical structure is referred to as a “derivative.”

Nucleotides are comprised of a nucleobase, a sugar, and at least one phosphate. Nucleotide can thus refer to nucleoside triphosphates, the substrates of RNA and DNA polymerases, nucleoside diphosphates, or nucleoside monophosphates, of which DNA and RNA are comprised. Nucleotides encompasses naturally occurring nucleotides or unnatural nucleotides (i.e., nucleotide analogs). Naturally occurring nucleotides include nucleotides found in naturally occurring DNA or RNA, including naturally occurring deoxyribonucleotides and ribonucleotides. Unnatural nucleotides contain some type of difference from the nucleobase, sugar, and/or phosphate moieties in naturally occurring nucleotides. A modified nucleotide comprises modification of one or more of the 3′OH or 5′OH group, the backbone, the sugar component, or the nucleobase, and/or addition of non-naturally occurring linker molecules. Unnatural nucleotides include DNA or RNA analogs (e.g., containing nucleobase analogs, sugar analogs and/or a non-native backbone and the like).

In some embodiments, a “nucleoside” is a compound comprising a nucleobase moiety and a sugar moiety. Nucleosides include, but are not limited to, naturally occurring nucleosides (corresponding to the nucleotides found in DNA and RNA), modified nucleosides, and nucleosides having mimetic nucleobases and/or sugar groups. Nucleosides include nucleosides comprising any variety of substituents. A nucleoside can be a glycoside compound formed through glycosidic linking between a nucleobase and a reducing group of a sugar.

A “nucleobase” is generally the heterocyclic portion of a nucleoside, and may be aromatic or partially unsaturated. The nucleobase does not include the sugar component of the nucleoside or nucleotide (e.g., ribose, deoxyribose, or analog thereof; examples of sugar analogs, also referred to as modified sugars, are described elsewhere herein). Nucleobases may be naturally occurring, may be modified, may bear no similarity to natural nucleobases, and may be synthesized, e.g., by organic synthesis. In certain embodiments, a nucleobase comprises any atom or group of atoms capable of interacting with a nucleobase of another nucleic acid with or without the use of hydrogen bonds. In certain embodiments, an unnatural nucleobase is not derived from a natural nucleobase. It should be noted that unnatural nucleobases do not necessarily possess basic properties; however, they are referred to as nucleobases for simplicity. In some embodiments, when referring to a nucleobase, a “(d)” indicates that the nucleobase can be attached to a deoxyribose or a ribose. Nucleobases are also commonly referred to as bases.

In some embodiments, the unnatural mRNA codons and unnatural tRNA anticodons as described in the present disclosure can be written in terms of their DNA coding sequence. For example, an unnatural tRNA anticodon can be written as GYU or GYT.

A “polynucleotide,” as the terms are used herein, refer to DNA, RNA, DNA- or RNA-like polymers such as peptide nucleic acids (PNA), locked nucleic acids (LNA), phosphorothioates, unnatural bases, and the like, which are well-known in the art. Polynucleotides can be synthesized in automated synthesizers, e.g., using phosphoroamidite chemistry or other chemical approaches adapted for synthesizer use.

“DNA” includes, but is not limited to, cDNA and genomic DNA. DNA may be attached, by covalent or non-covalent means, to another biomolecule, including, but not limited to, RNA or a peptide. “RNA” includes coding RNA, e.g. messenger RNA (mRNA). In some embodiments, RNA is rRNA, RNAi, snoRNA, microRNA, siRNA, snRNA, exRNA, piRNA, long ncRNA, or any combination or hybrid thereof. In some instances, RNA is a component of a ribozyme. DNA and RNA can be in any form, including, but not limited to, linear, circular, supercoiled, single-stranded, and double-stranded.

An “mRNA” is an RNA comprising an ORF capable of being translated by a ribosome.

A “tRNA” is an RNA capable of being charged with a natural amino acid or a ncAA and participating in translation of an mRNA by a ribosome.

A peptide nucleic acid (PNA) is a synthetic DNA/RNA analog wherein a peptide-like backbone replaces the sugar-phosphate backbone of DNA or RNA. PNA oligomers show higher binding strength and greater specificity in binding to complementary DNAs, with a PNA/DNA base mismatch being more destabilizing than a similar mismatch in a DNA/DNA duplex. This binding strength and specificity also applies to PNA/RNA duplexes. PNAs are not easily recognized by either nucleases or proteases, making them resistant to enzyme degradation. PNAs are also stable over a wide pH range. See also Nielsen P E, Egholm M, Berg R H, Buchardt O (December 1991). “Sequence-selective recognition of DNA by strand displacement with a thymine-substituted polyamide,” Science 254 (5037): 1497-500. doi:10.1126/science.1962210. PMID 1962210; and, Egholm M, Buchardt O, Christensen L, Behrens C, Freier S M, Driver D A, Berg R H, Kim S K, Nordén B, and Nielsen P E (1993), “PNA Hybridizes to Complementary Oligonucleotides Obeying the Watson-Crick Hydrogen Bonding Rules”. Nature 365 (6446): 566-8. doi:10.1038/365566a0. PMID 7692304

A locked nucleic acid (LNA) is a modified RNA nucleotide, wherein the ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. Such oligomers can be synthesized chemically and are commercially available. The locked ribose conformation enhances nucleobase stacking and backbone pre-organization. See, for example, Kaur, H; Arora, A; Wengel, J; Maiti, S (2006), “Thermodynamic, Counterion, and Hydration Effects for the Incorporation of Locked Nucleic Acid Nucleotides into DNA Duplexes”, Biochemistry 45 (23): 7347-55. doi:10.1021/bi060307w. PMID 16752924; Owczarzy R.; You Y., Groth C. L., Tataurov A. V. (2011), “Stability and mismatch discrimination of locked nucleic acid-DNA duplexes.”, Biochem. 50 (43): 9352-9367. doi:10.1021/bi200904e. PMC 3201676. PMID 21928795; Alexei A. Koshkin; Sanjay K. Singh, Poul Nielsen, Vivek K. Rajwanshi, Ravindra Kumar, Michael Meldgaard, Carl Erik Olsen, Jesper Wengel (1998), “LNA (Locked Nucleic Acids): Synthesis of the adenine, cytosine, guanine, 5-methylcytosine, thymine and uracil bicyclonucleoside monomers, oligomerisation, and unprecedented nucleic acid recognition”, Tetrahedron 54 (14): 3607-30. doi:10.1016/50040-4020(98)00094-5; and, Satoshi Obika; Daishu Nanbu, Yoshiyuki Hari, Ken-ichiro Morio, Yasuko In, Toshimasa Ishida, Takeshi Imanishi (1997), “Synthesis of 2′-0,4′-C-methyleneuridine and -cytidine. Novel bicyclic nucleosides having a fixed C3′-endo sugar puckering”, Tetrahedron Lett. 38 (50): 8735-8. doi:10.1016/S0040-4039(97)10322-7.

An “aptamer” refers an oligonucleotide that can specifically bind a target, e.g., with high affinity. Aptamers may comprise RNA and may comprise natural or unnatural nucleotides.

As used herein, “full length” means that a polynucleotide such as a cDNA is non-truncated relative to the complementary sequence that templated its synthesis (template polynucleotide). Where the template polynucleotide comprises an unnatural nucleotide, the full length polynucleotide comprises a nucleotide in the position complementary to the unnatural nucleotide in the template polynucleotide and further nucleotides 3′ thereof. A full length polynucleotide is in contrast to a truncated polynucleotide, which results from termination of synthesis before completion, e.g., at or near the position complementary to the unnatural nucleotide in the template polynucleotide.

The section headings used herein are for organizational purposes only and are not to be construed as limiting the subject matter described.

### Methods of Reverse Transcribing a Polynucleotide Comprising an Unnatural Ribonucleotide

Disclosed herein are methods of reverse transcribing a polynucleotide comprising an unnatural ribonucleotide. In such methods, the polynucleotide can be reverse transcribed with a reverse transcriptase in the presence of an unnatural dNTP comprising an unnatural nucleobase. The reverse transcriptase polymerizes cDNA into which the unnatural NTP is incorporated, e.g., in a position of the cDNA complementary to the position of the unnatural ribonucleotide in the polynucleotide.

In some embodiments, the polynucleotide is present at a concentration less than or equal to about 500 nM. In some embodiments, the RNA or polynucleotide is present during reverse transcription at a concentration in the range of about 1-10 nM, about 10-20 nM, about 20-30 nM, about 30-40 nM, about 40-50 nM, about 50-75 nM, about 75-100 nM, about 100-150 nM, about 150-200 nM, about 200-300 nM, about 300-400 nM, or about 400-500 nM. In some embodiments, the concentration is at or below about 100 nM, e.g., about 5-100 nM, such as about 10-100 nM. In some embodiments, the concentration is at or below about 50 nM, e.g., about 5-50 nM, such as about 10-50 nM. In some embodiments, the concentration is at or below about 30 nM, e.g., about 5-30 nM, such as about 10-30 nM. As described in the examples, using a lower concentration than previous attempts to reverse transcribe polynucleotides comprising an unnatural nucleotide may improve performance of the reverse transcription reaction.

Commercially available reverse transcriptases may be used in the disclosed methods. In some embodiments, the reverse transcriptase is Avian Myeloblastosis Virus (AMV) reverse transcriptase, Moloney Murine Leukemia Virus (MMLV) reverse transcriptase, Super Script II (SS II) reverse transcriptase, Super Script III (SS III) reverse transcriptase, Super Script IV (SS IV) reverse transcriptase, or Volcano 2G (V2G) reverse transcriptase. In some embodiments, the reverse transcriptase is SuperScript III (e.g., available from ThermoFisher Scientific, Cat. No. 18080093). SuperScript III is a genetically engineered MMLV reverse transcriptase that was created by introduction of several mutations for reduced RNase H activity, increased half-life, and improved thermal stability.

The polynucleotide comprising the unnatural ribonucleotide can be any suitable substrate for the reverse transcriptase, e.g., RNA, an RNA-DNA fusion, or DNA. Reverse transcriptases are known to accept DNA or RNA-DNA hybrids as substrates in addition to RNA. In some embodiments, the polynucleotide comprising the unnatural ribonucleotide is an RNA. For example, the RNA can be an mRNA. In another example, the RNA can be a tRNA. In a still further example, the RNA can be an RNA aptamer, or a member of a plurality of aptamer candidates (often referred to as a “library”), e.g., wherein the plurality of aptamer candidates undergoes reverse transcription in the same or different reaction vessels or chambers. The polynucleotide(s) in any of the foregoing embodiments may comprise other modifications in addition to the unnatural nucleotide; for example, there can be an unnatural nucleotide comprising an unnatural nucleobase and, at the same and/or other nucleotide positions, modifications to the nucleobase or one or more sugars and/or phosphates.

Where the RNA is an mRNA, the unnatural ribonucleotide may be located in a codon. The unnatural nucleotide may occur in the first, second, or third position of the codon. Exemplary codons are AXC, AYC, GXC, GYC, GXT, GYT, AXA, AXT, TXA, or TXT, where the unnatural ribonucleotide may be represented by X or Y. In some embodiments, X comprises

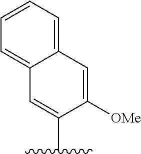 [SMILES: *c1cc2ccccc2cc1OC]

as the nucleobase of the unnatural ribonucleotide (NaM; here and throughout, for clarity only the nucleobase portion of the unnatural deoxy- or ribonucleotide/nucleoside is shown) and/or Y comprises

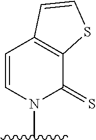 [SMILES: *n1ccc2ccsc2c1=S]

as the nucleobase of the unnatural ribonucleotide (TPT3).

Where the RNA is a tRNA, the unnatural ribonucleotide may be located in the anticodon of the tRNA. The unnatural nucleotide may occur in the first, second, or third position of the anticodon. Exemplary anticodons are GYT, GXT, GYC, GXC, CYA, CXA, AYC, or AXC, where the unnatural ribonucleotide may be represented by X or Y. In some embodiments, X comprises

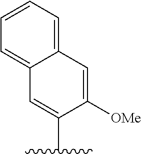 [SMILES: *c1cc2ccccc2cc1OC]

as the nucleobase of the unnatural ribonucleotide (NaM) and/or Y comprise:

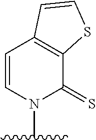 [SMILES: *n1ccc2ccsc2c1=S]

as the nucleobase of the unnatural ribonucleotide (TPT3).

Various unnatural nucleobases are known and can be used as the unnatural nucleobase in the dNTP and/or the unnatural ribonucleotide. In some embodiments, the unnatural nucleobase is independently selected from a group consisting of:

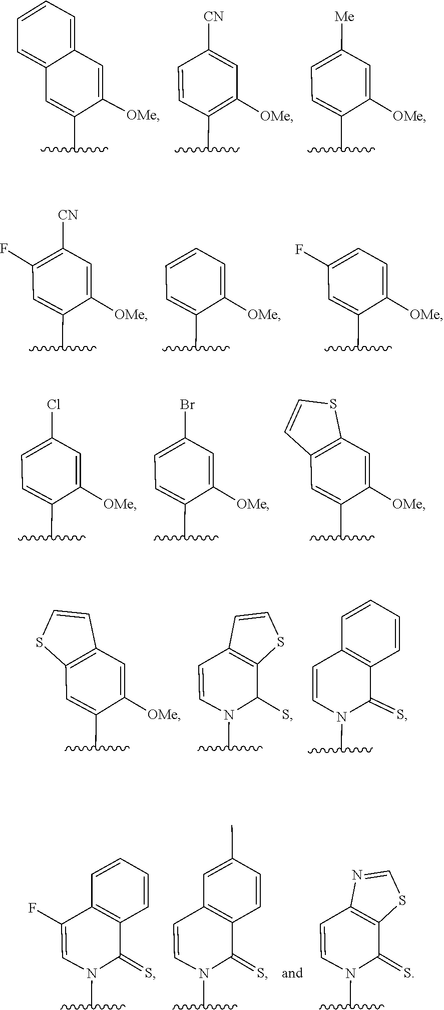

In some embodiments, the unnatural dNTP is not dTPT3TP.

In some embodiments, the unnatural nucleobase is selected from those shown below, wherein the wavy line or R identifies a point of attachment to the sugar (e.g., deoxyribose or ribose):

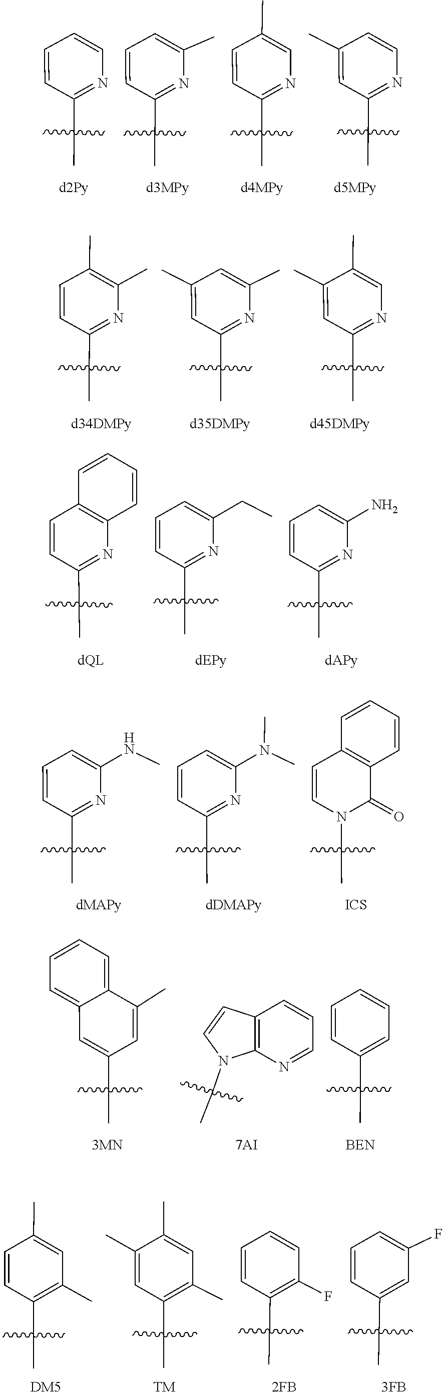

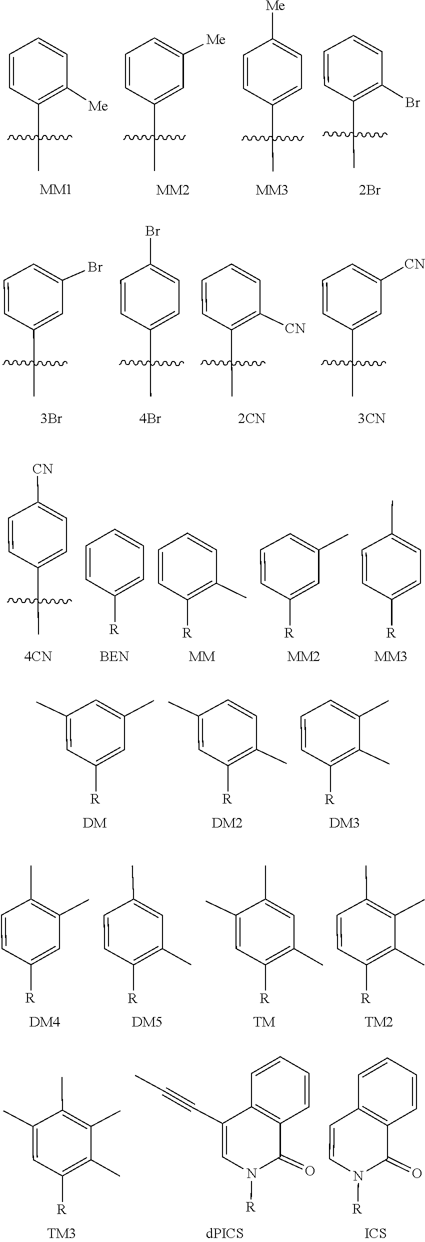

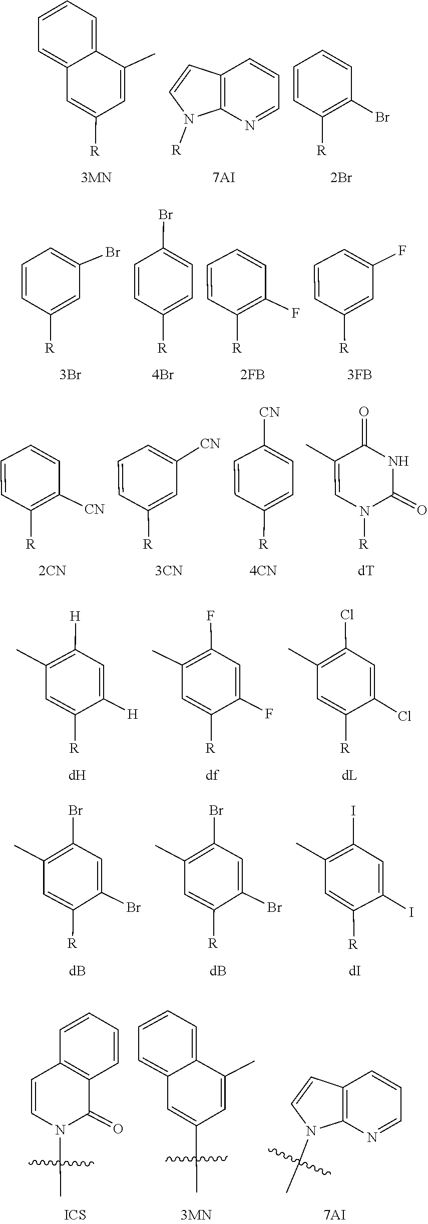

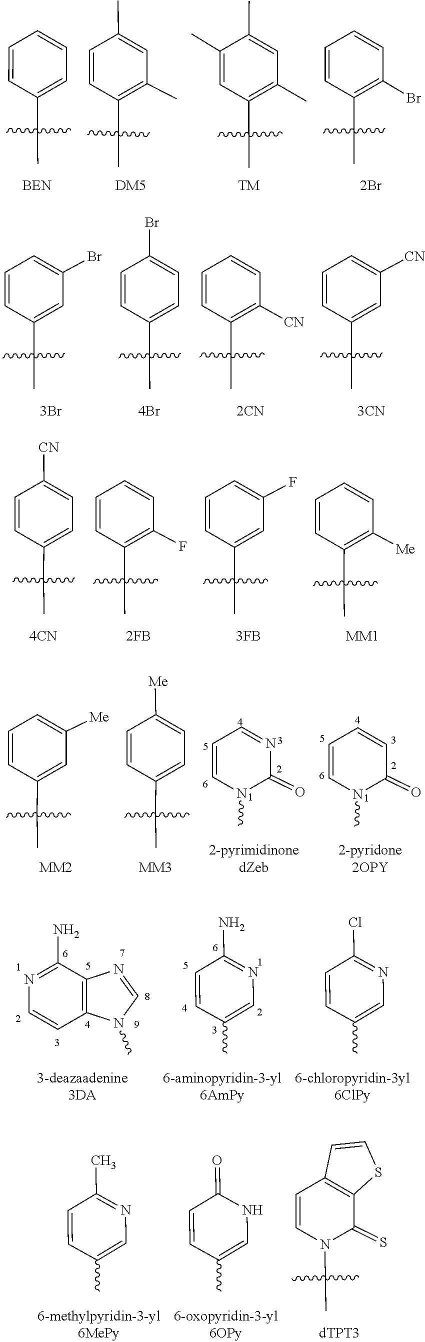

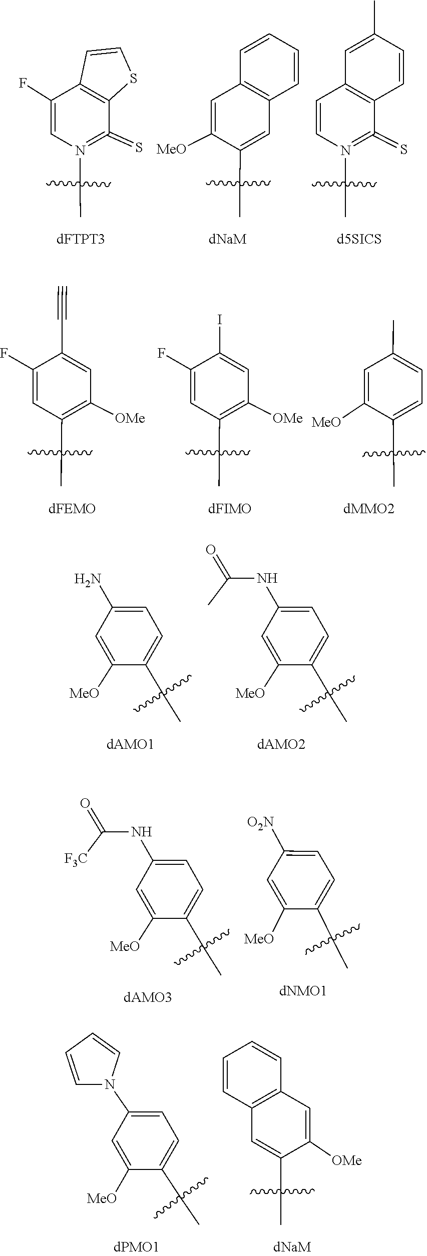

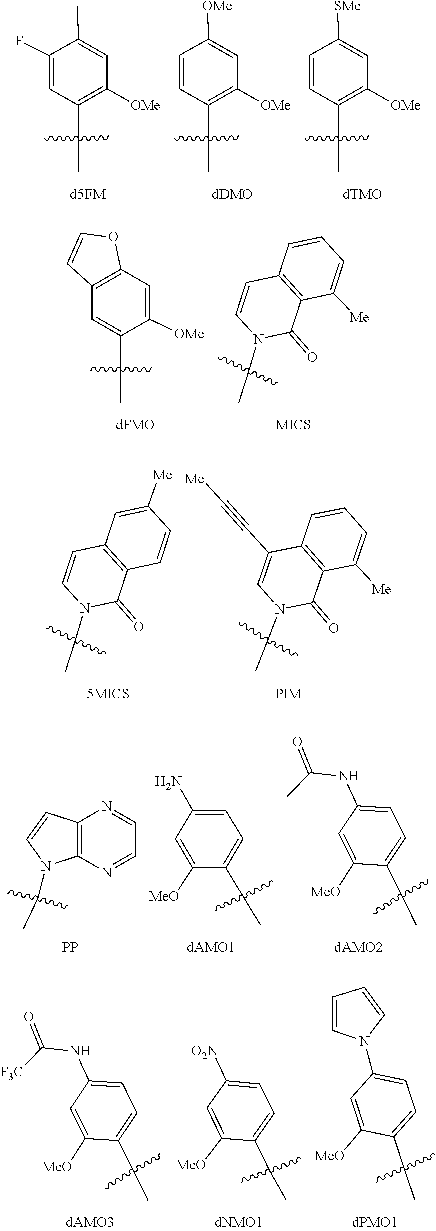

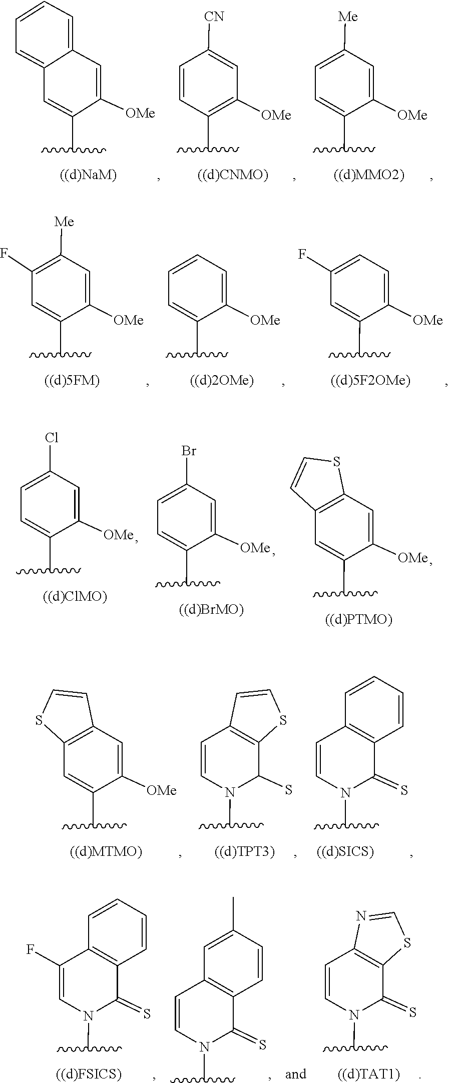

In some embodiments, the nucleobase comprises the structure:

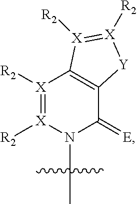

wherein each X is independently carbon or nitrogen; R₂ is optional and when present is independently hydrogen, alkyl, alkenyl, alkynyl; methoxy, methanethiol, methaneseleno, halogen, cyano, or azide group; wherein each Y is independently sulfur, oxygen, selenium, or secondary amine; wherein each E is independently oxygen, sulfur or selenium; and wherein the wavy line indicates a point of bonding to a ribosyl, deoxyribosyl, or dideoxyribosyl moiety or an analog thereof, wherein the ribosyl, deoxyribosyl, or dideoxyribosyl moiety or analog thereof is in free form, connected to a mono-phosphate, diphosphate, or triphosphate group, optionally comprising an α-thiotriphosphate, β-thiotriphosphate, or γ-thiotriphosphate group, or is included in an RNA or a DNA or in an RNA analog or a DNA analog. In some embodiments, R₂ is lower alkyl (e.g., C₁-C₆), hydrogen, or halogen. In some embodiments of a nucleobase described herein, R₂ is fluoro. In some embodiments of a nucleobase described herein, X is carbon. In some embodiments of a nucleobase described herein, E is sulfur. In some embodiments of a nucleobase described herein, Y is sulfur. In some embodiments of a nucleobase described herein, a nucleobase has the structure:

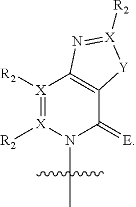

In some embodiments of a nucleobase described herein, E is sulfur and Y is sulfur. In some embodiments of a nucleobase described herein, the wavy line indicates a point of bonding to a ribosyl or deoxyribosyl moiety. In some embodiments of a nucleobase described herein, the wavy line indicates a point of bonding to a ribosyl or deoxyribosyl moiety, connected to a triphosphate group.

In some embodiments the nucleobase is a component of a nucleic acid polymer. In some embodiments, the nucleobase is a component of a tRNA. In some embodiments, the nucleobase is a component of an anticodon in a tRNA. In some embodiments, the nucleobase is a component of an mRNA. In some embodiments, the nucleobase is a component of a codon of an mRNA. In some embodiments, the nucleobase is a component of RNA or DNA. In some embodiments, the nucleobase is a component of a codon in DNA. In some embodiments, the nucleobase forms a nucleobase pair with another complementary nucleobase.

Additional examples of unnatural nucleobases include 2-thiouracil, 2′-deoxyuridine, 4-thio-uracil, uracil-5-yl, hypoxanthin-9-yl (I), 5-halouracil; 5-propynyl-uracil, 6-azo-uracil, 5-methylaminomethyluracil, 5-methoxyaminomethyl-2-thiouracil, pseudouracil, uracil-5-oxacetic acid methylester, uracil-5-oxacetic acid, 5-methyl-2-thiouracil, 3-(3-amino-3-N-2-carboxypropyl) uracil, 5-methyl-2-thiouracil, 4-thiouracil, 5-methyluracil, 5′-methoxycarboxymethyluracil, 5-methoxyuracil, uracil-5-oxyacetic acid, 5-(carboxyhydroxylmethyl) uracil, 5-carboxymethylaminomethyl-2-thiouridine, 5-carboxymethylaminomethyluracil, dihydrouracil, 5-hydroxymethyl cytosine, 5-trifluoromethyl cytosine, 5-halocytosine, 5-propynyl cytosine, 5-hydroxycytosine, cyclocytosine, cytosine arabinoside, 5,6-dihydrocytosine, 5-nitrocytosine, 6-azo cytosine, azacytosine, N4-ethylcytosine, 3-methylcytosine, 5-methylcytosine, 4-acetylcytosine, 2-thiocytosine, phenoxazine cytidine([5,4-b][1,4]benzoxazin-2(3H)-one), phenothiazine cytidine (1H-pyrimido[5,4-b][1, 4]benzothiazin-2(3H)-one), phenoxazine cytidine (9-(2-aminoethoxy)-H-pyrimido[5,4-b][1,4]benzoxazin-2(3H)-one), carbazole cytidine (2H-pyrimido[4,5-b]indo1-2-one), pyridoindole cytidine (H-pyrido [3′,2′:4,5]pyrrolo [2,3-d]pyrimidin-2-one), 2-aminoadenine, 2-propyl adenine, 2-amino-adenine, 2-F-adenine, 2-amino-propyl-adenine, 2-amino-2′-deoxyadenosine, 3-deazaadenine, 7-methyladenine, 7-deaza-adenine, 8-azaadenine, 8-halo, 8-amino, 8-thiol, 8-thioalkyl, and 8-hydroxyl substituted adenines, N6-isopentenyladenine, 2-methyladenine, 2,6-diaminopurine, 2-methythio-N6-isopentenyladenine, 6-aza-adenine, 2-methylguanine, 2-propyl and alkyl derivatives of guanine, 3-deazaguanine, 6-thio-guanine, 7-methylguanine, 7-deazaguanine, 7-deazaguanosine, 7-deaza-8-azaguanine, 8-azaguanine, 8-halo, 8-amino, 8-thiol, 8-thioalkyl, and 8-hydroxyl substituted guanines, 1-methylguanine, 2,2-dimethylguanine, 7-methylguanine, 6-aza-guanine, hypoxanthine, xanthine, 1-methylinosine, queosine, beta-D-galactosylqueosine, inosine, beta-D-mannosylqueosine, wybutoxosine, hydroxyurea, (acp3)w, 2-aminopyridine, or 2-pyridone.

In some embodiments, the unnatural nucleobase is selected from uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, O-6 substituted purines, 2-aminopropyladenine, 5-propynyluracil, 5-propynylcytosine, 5-methylcytosine, those that increase the stability of duplex formation, universal nucleic acids, hydrophobic nucleobases, promiscuous nucleobases, size-expanded nucleobases, fluorinated nucleobases, 5-substituted pyrimidines, 6-azapyrimidines and N-2, N-6 and O-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—CC—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5 -halo particularly 5-bromo, 5-trifluoromethyl, other 5-substituted uracils and cytosines, 7-methylguanine, 7- methyladenine, 2-F-adenine, 2-amino-adenine, 8-azaguanine, 8-azaadenine, 7-deazaguanine, 7- deazaadenine, 3-deazaguanine, 3-deazaadenine, tricyclic pyrimidines, phenoxazine cytidine([5,4-b][1,4]benzoxazin-2(3H)-one), phenothiazine cytidine (1H- pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5- b]indo1-2-one), pyridoindole cytidine (H-pyrido[3′,2′:4,5]pyrrolo[2,3-d]pyrimidin-2-one), those in which the purine or pyrimidine nucleobase is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 4-thio-uracil, N4-ethylcytosine, 7-deazaguanine, 7-deaza-8- azaguanine, 5-hydroxycytosine, 2′-deoxyuridine, 2-amino-2′-deoxyadenosine, and those described in U.S. Pat. Nos. 3,687,808; 4,845,205; 4,910,300; 4,948,882; 5,093,232; 5,130,302; 5,134,066; 5,367,066; 5,432,272; 5,457,187; 5,459,255; 5,484,908; 5,502,177; 5,525,711; 5,587,469; 5,594,121; 5,596,091; 5,614,617; 5,645,985; 5,681,941; 5,750,692; 5,830,653 and 6,005,096; WO 99/62923; Kandimalla et al., (2001) Bioorg. Med. Chem. 9:807-813; The Concise Encyclopedia of Polymer Science and Engineering, Kroschwitz, J. I., Ed., John Wiley & Sons, 1990, 858-859; Englisch et al., Angewandte Chemie, International Edition, 1991, 30, 613; and Sanghvi, Chapter 15, Antisense Research and Applications, Crooke and Lebleu Eds., CRC Press, 1993, 273-288. Additional nucleobase modifications can be found, for example, in U.S. Pat. No. 3,687,808; Englisch et al., Angewandte Chemie, International Edition, 1991, 30, 613.

Unnatural nucleic acids comprising various heterocyclic nucleobases and various sugar moieties (and sugar analogs) are available in the art, and the nucleic acid in some cases include one or several heterocyclic nucleobases other than the principal five nucleobase components of naturally-occurring nucleic acids. For example, the heterocyclic nucleobase includes, in some cases, uracil-5-yl, cytosin-5-yl, adenin-7-yl, adenin-8-yl, guanin-7-yl, guanin-8-yl, 4-aminopyrrolo [2.3-d] pyrimidin-5-yl, 2-amino-4-oxopyrolo [2,3-d] pyrimidin-5-yl, 2-amino-4-oxopyrrolo [2.3-d] pyrimidin-3-yl groups, where the purines are attached to the sugar moiety of the nucleic acid via the 9-position, the pyrimidines via the 1-position, the pyrrolopyrimidines via the 7-position and the pyrazolopyrimidines via the 1-position.

In some embodiments, nucleotide analogs are also modified at the phosphate moiety. Modified phosphate moieties include, but are not limited to, those with modification at the linkage between two nucleotides and contains, for example, a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides are through a 3′-5′ linkage or a 2′-5′ linkage, and the linkage contains inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,278,302; 5,286,717; 5,321,131; 5,399,676; 5,405,939; 5,453,496; 5,455,233; 5,476,925; 5,519,126; 5,536,821; 5,541,306; 5,550,111; 5,563,253; 5,571,799; and 5,625,050.

In some embodiments, unnatural nucleic acids include 2′,3′-dideoxy-2′,3′-didehydro-nucleosides (PCT/US2002/006460), 5′-substituted DNA and RNA derivatives (PCT/US2011/033961; Saha et al., J. Org Chem., 1995, 60, 788-789; Wang et al., Bioorganic & Medicinal Chemistry Letters, 1999, 9, 885-890; and Mikhailov et al., Nucleosides & Nucleotides, 1991, 10(1-3), 339-343; Leonid et al., 1995, 14(3-5), 901-905; and Eppacher et al., Helvetica Chimica Acta, 2004, 87, 3004-3020; PCT/JP2000/004720; PCT/JP2003/002342; PCT/JP2004/013216; PCT/JP2005/020435; PCT/JP2006/315479; PCT/JP2006/324484; PCT/JP2009/056718; PCT/JP2010/067560), or 5′-substituted monomers made as the monophosphate with modified nucleobases (Wang et al., Nucleosides Nucleotides & Nucleic Acids, 2004, 23 (1 & 2), 317-337).

In some embodiments, unnatural nucleic acids include modifications at the 5′-position and the 2′-position of the sugar ring (PCT/US94/02993), such as 5′-CH₂-substituted 2′-O-protected nucleosides (Wu et al., Helvetica Chimica Acta, 2000, 83, 1127-1143 and Wu et al., Bioconjugate Chem. 1999, 10, 921-924). In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃ (Mesmaeker et al., Synlett, 1997, 1287-1290). Unnatural nucleic acids can include 2′-substituted 5′-CH₂ (or modified nucleosides (PCT/US92/01020). Unnatural nucleic acids can include 5′-methylenephosphonate DNA and RNA monomers, and dimers (Bohringer et al., Tet. Lett., 1993, 34, 2723-2726; Collingwood et al., Synlett, 1995, 7, 703-705; and Hutter et al., Helvetica Chimica Acta, 2002, 85, 2777-2806). Unnatural nucleic acids can include 5′-phosphonate monomers having a 2′-substitution (US2006/0074035) and other modified 5′-phosphonate monomers (WO1997/35869). Unnatural nucleic acids can include 5′-modified methylenephosphonate monomers (EP614907 and EP629633). Unnatural nucleic acids can include analogs of 5′ or 6′-phosphonate ribonucleosides comprising a hydroxyl group at the 5′ and/or 6′-position (Chen et al., Phosphorus, Sulfur and Silicon, 2002, 777, 1783-1786; Jung et al., Bioorg. Med. Chem., 2000, 8, 2501-2509; Gallier et al., Eur. J. Org. Chem., 2007, 925-933; and Hampton et al., J. Med. Chem., 1976, 19(8), 1029-1033). Unnatural nucleic acids can include 5′-phosphonate deoxyribonucleoside monomers and dimers having a 5′-phosphate group (Nawrot et al., Oligonucleotides, 2006, 16(1), 68-82). Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof) (Fairhurst et al., Synlett, 2001, 4, 467-472; Kappler et al., J. Med. Chem., 1986, 29, 1030-1038; Kappler et al., J. Med. Chem., 1982, 25, 1179-1184; Vrudhula et al., J. Med. Chem., 1987, 30, 888-894; Hampton et al., J. Med. Chem., 1976, 19, 1371-1377; Geze et al., J. Am. Chem. Soc, 1983, 105(26), 7638-7640; and Hampton et al., J. Am. Chem. Soc, 1973, 95(13), 4404-4414).

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids (BNA); replacement of the ribosyl ring oxygen atom with S, N(R), or C(R₁)(R₂) (R═H, C₁-C₁₂ alkyl or a protecting group); and combinations thereof. Examples of chemically modified sugars can be found in WO2008/101157, US2005/0130923, and WO2007/134181.

In some instances, a modified nucleic acid comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety may be the furanoside of ribose, deoxyribose, arabinose or 2′-O-alkylribose, and the sugar can be attached to the respective heterocyclic nucleobases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol like ribonucleosides. The preparation of these sugars or sugar analogs and the respective “nucleosides” wherein such sugars or analogs are attached to a heterocyclic nucleobase (nucleic acid base) is known. Sugar modifications may also be made and combined with other modifications.

Modifications to the sugar moiety include natural modifications of the ribose and deoxy ribose as well as unnatural modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]ₘ CH₃, —O(CH₂)ₙOCH₃, —O(CH₂)ₙNH₂, —O(CH₂)ₙCH₃, —O(CH₂)ₙONH₂, and —O(CH₂)ₙON[CH₂)n CH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Modified sugars also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures and which detail and describe a range of nucleobase modifications, such as U.S. Pat. Nos. 4,981,957; 5,118,800; 5,319,080; 5,359,044; 5,393,878; 5,466,786; 5,514,785; 5,519,134; 5,567,811; 5,576,427; 5,591,722; 5,597,909; 5,627,053; 5,639,873; 5,646,265; 5,658,873; 5,670,633; 4,845,205; 5,130,302; 5,175,273; 5,367,066; 5,432,272; 5,457,187; 5,459,255; 5,484,908; 5,502,177; 5,552,540; 5,587,469; 5,594,121, 5,596,091; 5,614,617; 5,681,941; and 5,700,920, each of which is herein incorporated by reference in its entirety.

Examples of nucleic acids having modified sugar moieties include, without limitation, nucleic acids comprising 5′-vinyl, 5′-methyl (R or S), 4′-S, 2′-F, 2′-OCH₃, and 2′-substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′, and analogs thereof (see, U.S. Pat. No. 7,399,845); 4′-C(CH₃)(CH₃)—O-2′and analogs thereof, (see WO2009/006478, WO2008/150729, US2004/0171570, U.S. Pat. No. 7,427,672, Chattopadhyaya et al., J. Org. Chem., 209, 74, 118-134, and WO2008/154401). Also see, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invens. Drugs, 2001, 2, 558-561; Braasch et al., Chem. Biol, 2001, 8, 1-7; Oram et al., Curr. Opinion Mol. Ther., 2001, 3, 239-243; U.S. Pat. Nos. 4,849,513; 5,015,733; 5,118,800; 5,118,802; 7,053,207; 6,268,490; 6,770,748; 6,794,499; 7,034,133; 6,525,191; 6,670,461; and 7,399,845; International Publication Nos. WO2004/106356, WO1994/14226, WO2005/021570, WO2007/090071, and WO2007/134181; U.S. Patent Publication Nos. US2004/0171570, US2007/0287831, and US2008/0039618; U.S. Provisional Application Nos. 60/989,574, 61/026,995, 61/026,998, 61/056,564, 61/086,231, 61/097,787, and 61/099,844; and International Applications Nos. PCT/US2008/064591, PCT US2008/066154, PCT US2008/068922, and PCT/DK98/00393.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and may be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety and can be a monophosphate, diphosphate, triphosphate, alkylphosphonate, phosphorothioate, phosphorodithioate, phosphoramidate or the like. Exemplary polynucleotides containing modified phosphate linkages or non-phosphate linkages can be found in Peyrottes et al., 1996, Nucleic Acids Res. 24: 1841-1848; Chaturvedi et al., 1996, Nucleic Acids Res. 24:2318-2323; and Schultz et al., (1996) Nucleic Acids Res. 24:2966-2973; Matteucci, 1997, “Oligonucleotide Analogs: an Overview” in Oligonucleotides as Therapeutic Agents, (Chadwick and Cardew, ed.) John Wiley and Sons, New York, NY; Zon, 1993, “Oligonucleoside Phosphorothioates” in Protocols for Oligonucleotides and Analogs, Synthesis and Properties, Humana Press, pp. 165-190; Miller et al., 1971, JACS 93:6657-6665; Jager et al., 1988, Biochem. 27:7247-7246; Nelson et al., 1997, JOC 62:7278-7287; U.S. Pat. No. 5,453,496; and Micklefield, 2001, Curr. Med. Chem. 8: 1157-1179.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos (Micklefield, 2001, Current Medicinal Chemistry 8: 1157-1179). A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,185,444; 5,214,134; 5,216,141; 5,235,033; 5,264,562; 5,264,564; 5,405,938; 5,466,677; 5,470,967; 5,489,677; 5,541,307; 5,561,225; 5,596,086; 5,602,240; 5,602,240; 5,608,046; 5,610,289; 5,618,704; 5,623,070; 5,663,312; 5,633,360; and 5,677,439. It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. See also Nielsen et al., Science, 1991, 254, 1497-1500. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. KY. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EM5OJ, 1991, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1-di-O-hexadecyl-rac-glycero-S—H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochem. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). Numerous United States patents teach the preparation of such conjugates and include, but are not limited to U.S. Pat. Nos. 4,828,979; 4,948,882; 5,218,105; 5,525,465; 5,541,313; 5,552,538; 5,578,717, 5,580,731; 5,580,731; 5,591,584; 5,109,124; 5,118,802; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,112,963; 5,214,136; 5,082,830; 5,112,963; 5,214,136; 5,245,022; 5,254,469; 5,262,536; 5,272,250; 5,292,873; 5,317,098; 5,371,241, 5,391,723; 5,416,203, 5,510,475; 5,512,667; 5,514,785; 5,565,552; 5,567,810; 5,574,142; 5,585,481; 5,595,726; 5,597,696; 5,599,923; 5,599,928 and 5,688,941.

In some embodiments, a polynucleotide (also referred to as a nucleic acid) comprising an unnatural ribonucleotide is from any source or composition, such as DNA, cDNA, gDNA (genomic DNA), RNA, siRNA (short inhibitory RNA), RNAi, tRNA, mRNA or rRNA (ribosomal RNA), for example, and is in any form (e.g., linear, circular, supercoiled, single-stranded, double-stranded, and the like). In some embodiments, nucleic acids comprise nucleotides, nucleosides, or polynucleotides. In some cases, nucleic acids comprise natural and unnatural nucleic acids. In some cases, a nucleic acid also comprises unnatural nucleic acids, such as DNA or RNA analogs (e.g., containing nucleobase analogs, sugar analogs and/or a non-native backbone and the like). It is understood that the term “nucleic acid” does not refer to or infer a specific length of the polynucleotide chain, thus polynucleotides and oligonucleotides are also included in the definition. A nucleic acid sometimes is a vector, plasmid, phagemid, autonomously replicating sequence (ARS), centromere, artificial chromosome, yeast artificial chromosome (e.g., YAC) or other nucleic acid able to replicate or be replicated in a host cell. In some cases, an unnatural nucleic acid is a nucleic acid analogue. In additional cases, an unnatural nucleic acid is from an extracellular source. In other cases, an unnatural nucleic acid is available to the intracellular space of an organism provided herein, e.g., a genetically modified organism. In some embodiments, an unnatural nucleotide is not a natural nucleotide. In some embodiments, a nucleotide that does not comprise a natural nucleobase comprises an unnatural nucleobase.

In some embodiments polynucleotides are used as a substrate for an reverse transcriptase or synthesized by a reverse transcriptase comprising natural nucleotides in addition to at least one unnatural nucleotide. Exemplary natural nucleotides include, without limitation, ATP, UTP, CTP, GTP, ADP, UDP, CDP, GDP, AMP, UMP, CMP, GMP, dATP, dTTP, dCTP, dGTP, dADP, dTDP, dCDP, dGDP, dAMP, dTMP, dCMP, and dGMP. Exemplary natural deoxyribonucleotides include dATP, dTTP, dCTP, dGTP, dADP, dTDP, dCDP, dGDP, dAMP, dTMP, dCMP, and dGMP. Exemplary natural ribonucleotides include ATP, UTP, CTP, GTP, ADP, UDP, CDP, GDP, AMP, UMP, CMP, and GMP. It is understood that triphosphate forms of nucleotides are the substrate for polymerization, and that upon addition to a nascent polynucleotide chain the nucleotide is converted to a nucleotide of the monophosphate form.

In general, a nucleotide analog, or unnatural nucleotide, comprises a nucleotide which contains some type of modification to either the nucleobase, sugar, or phosphate moieties. In some embodiments, a modification comprises a chemical modification. In some cases, modifications occur at the 3′OH or 5′OH group, at the backbone, at the sugar component, or at the nucleobase. In one aspect, the modified nucleic acid comprises modification of one or more of the 3′OH or 5′OH group, the backbone, the sugar component, or the nucleobase, and/or addition of non-naturally occurring linker molecules. In one aspect, a modified backbone comprises a backbone other than a phosphodiester backbone. In one aspect, a modified sugar comprises a sugar other than deoxyribose (in modified DNA) or other than ribose (modified RNA). In one aspect, a modified nucleobase comprises a nucleobase other than adenine, guanine, cytosine or thymine (in modified DNA) or a nucleobase other than adenine, guanine, cytosine or uracil (in modified RNA).

In some embodiments, the nucleic acid comprises at least one modified nucleobase. In some instances, the nucleic acid comprises 2, 3, 4, 5, 6, 7, 8, 9, 10, 15, 20, or more modified nucleobases. In some cases, modifications to the nucleobase moiety include natural and synthetic modifications of A, C, G, and T/U as well as different purine or pyrimidine nucleobases. In some embodiments, a modification is to a modified form of adenine, guanine cytosine or thymine (in modified DNA) or a modified form of adenine, guanine cytosine or uracil (modified RNA). The modified nucleobase may be any of the modified nucleobases specifically described elsewhere herein.

In some embodiments, the reverse transcriptase produces full-length cDNA. In some embodiments, the reverse transcriptase produces cDNA that comprises a nucleotide in the position complementary to the unnatural ribonucleotide in the polynucleotide undergoing reverse transcription and a plurality of nucleotides 3′ of the nucleotide in the position complementary to the unnatural ribonucleotide (e.g., at least 2, 5, 10, or 20 nucleotides) and includes cDNA that is fully complementary to the polynucleotide undergoing reverse transcription. In some embodiments, the cDNA comprises at least 90%, 95%, 97%, or 99% as many nucleotides as the polynucleotide undergoing reverse transcription. In some embodiments, the cDNA is fully complementary to the polynucleotide undergoing reverse transcription. In some embodiments, at least 25% of the cDNA comprises the unnatural nucleobase. In some embodiments, at least 30%, 40%, 50%, 60%, 70%, 80%, 90%, 95%, 97%, 98%, or 99% of the cDNA comprises the unnatural nucleobase.

### Unnatural Base Pairs

In some embodiments, an unnatural nucleotide forms a base pair (an unnatural base pair; UBP) with another unnatural nucleotide during and/or after incorporation, e.g., by a reverse transcriptase. In some embodiments, a stably integrated unnatural nucleotide is an unnatural nucleotide that can form a base pair with another nucleotide, e.g., a natural or unnatural nucleotide. In some embodiments, a stably integrated unnatural nucleotide is an unnatural nucleotide that can form a base pair with another unnatural nucleotide (unnatural base pair (UBP)). For example, a first unnatural nucleotide can form a base pair with a second unnatural nucleotide. For example, one pair of unnatural nucleoside triphosphates that can base pair during and/or after incorporation into nucleic acids include a triphosphate of (d)5SICS ((d)5SICSTP) and a triphosphate of (d)NaM ((d)NaMTP). Other examples include but are not limited to: a triphosphate of (d)CNMO ((d)CNMOTP) and a triphosphate of (d)TPT3 ((d)TPT3TP). Such unnatural nucleotides can have a ribose or deoxyribose sugar moiety (indicated by the “(d)”). For example, one pair of unnatural nucleoside triphosphates that can base pair when incorporated into nucleic acids includes a triphosphate of (d)TAT1 ((d)TAT1TP) and a triphosphate of (d)NaM ((d)NaMTP). In some embodiments, one pair of unnatural nucleoside triphosphates that can base pair when incorporated into nucleic acids includes a triphosphate of (d)CNMO ((d)CNMOTP) and a triphosphate of (d)TAT1 ((d)TAT1TP). In some embodiments, one pair of unnatural nucleoside triphosphates that can base pair when incorporated into nucleic acids includes a triphosphate of (d)TPT3 ((d)TPT3TP) and a triphosphate of (d)NaM ((d)NaMTP). In some embodiments, an unnatural nucleotide does not substantially form a base pair with a natural nucleotide (A, T, G, C, U). In some embodiments, a stably integrated unnatural nucleotide can form a base pair with a natural nucleotide.

In some embodiments, a stably integrated unnatural (deoxy)ribonucleotide is an unnatural (deoxy)ribonucleotide that can form a UBP but does not substantially form a base pair with each any of the natural (deoxy)ribonucleotides. In some embodiments, a stably integrated unnatural (deoxy)ribonucleotide is an unnatural (deoxy)ribonucleotide that can form a UBP but does not substantially form a base pair with one or more natural nucleic acids. For example, a stably integrated unnatural nucleotide may not substantially form a base pair with A, T, and, C, but can form a base pair with G. For example, a stably integrated unnatural nucleotide may not substantially form a base pair with A, T, and, G, but can form a base pair with C. For example, a stably integrated unnatural nucleotide may not substantially form a base pair with C, G, and, A, but can form a base pair with T. For example, a stably integrated unnatural nucleotide may not substantially form a base pair with C, G, and, T, but can form a base pair with A. For example, a stably integrated unnatural nucleotide may not substantially form a base pair with A and T, but can form a base pair with C and G. For example, a stably integrated unnatural nucleotide may not substantially form a base pair with A and C, but can form a base pair with T and G. For example, a stably integrated unnatural nucleotide may not substantially form a base pair with A and G, but can form a base pair with C and T. For example, a stably integrated unnatural nucleotide may not substantially form a base pair with C and T, but can form a base pair with A and G. For example, a stably integrated unnatural nucleotide may not substantially form a base pair with C and G, but can form a base pair with T and G. For example, a stably integrated unnatural nucleotide may not substantially form a base pair with T and G, but can form a base pair with A and G. For example, a stably integrated unnatural nucleotide may not substantially form a base pair with, G, but can form a base pair with A, T, and, C. For example, a stably integrated unnatural nucleotide may not substantially form a base pair with, A, but can form a base pair with G, T, and, C. For example, a stably integrated unnatural nucleotide may not substantially form a base pair with, T, but can form a base pair with G, A, and, C. For example, a stably integrated unnatural nucleotide may not substantially form a base pair with, C, but can form a base pair with G, T, and, A.

Exemplary unnatural nucleotides capable of forming an unnatural DNA or RNA base pair (UBP) include, but are not limited to, (d)5SICS, (d)5SICS, (d)NaM, (d)NaM, (d)TPT3, (d)MTMO, (d)CNMO, (d)TAT1, and combinations thereof. In some embodiments, unnatural nucleotide base pairs include but are not limited to:

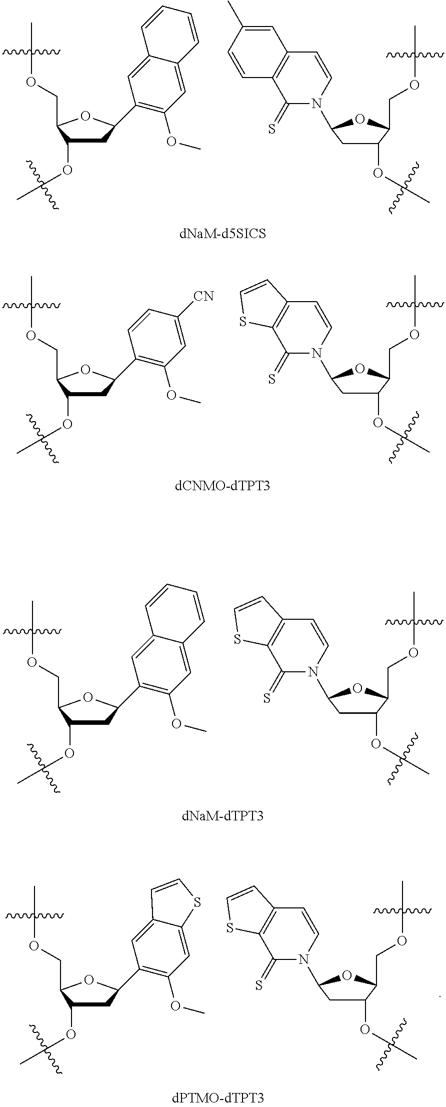

In some embodiments, such as where an RNA has undergone reverse transcription, a UBP is formed wherein the unnatural nucleobases are as shown above or described elsewhere herein and one of the sugars is a ribose or a modified form thereof (but is not deoxyribose).

### Measuring Unnatural Nucleotide Content in an Oligonucleotide

In some embodiments, methods disclosed herein comprise measuring the amount of an unnatural nucleotide, e.g., in a cDNA. Where the cDNA was produced from an RNA transcribed from a DNA molecule, such an approach can be used to determine, independently of translation, a lower bound for the fidelity of retention of an unnatural nucleotide during transcription. In some embodiments, the method is for measuring combined fidelity of transcription and reverse transcription. In some embodiments, the method is for measuring retention of an unnatural nucleotide during transcription and reverse transcription.

In some embodiments, the measuring step can use a binding partner can be used that recognizes an unnatural nucleobase. Where the unnatural nucleobase comprises a biotin moiety, the binding partner can be a biotin-binding agent (e.g., streptavidin, avidin, Neutravidin, or an anti-biotin antibody). In some embodiments, the biotin-binding agent is associated with (e.g., bound to, such as covalently) a solid support, such as beads. In some embodiments, the binding partner is streptavidin. Binding of the binding partner can be assessed in a gel shift assay or mobility shift assay, in that polynucleotide bound to the binding partner (understood to comprise the unnatural nucleobase) will exhibit a different electrophoretic mobility than unbound polynucleotide (understood to lack the unnatural nucleobase). Where the unnatural nucleobase of the nucleotide incorporated by a reverse transcriptase does not itself comprise a biotin moiety or other target for a binding partner, a binding partner can still be used to measure the amount of the unnatural nucleobase, e.g., as follows. A complementary molecule or amplicon can be generated from the cDNA (e.g., as described for biotin shift assays performed in the Examples) that does comprise a biotinylated unnatural nucleobase, which can then be assayed as a proxy for the cDNA, with appropriate adjustments in the calculations. In some embodiments, the amplification of the cDNA is by PCR. Exemplary biotinylated unnatural nucleobases can be incorporated in the complementary molecule or amplicon using dMMO2bioTP (a biotinylated analog of dNaMTP) and d5SICSTP (an analog of dTPT3TP that pairs with dMMO2bio during replication better than dTPT3TP itself (Malyshev et al., A Semi-Synthetic Organism with an Expanded Genetic Alphabet. Nature 2014, 509, 385-388.) Such an approach, in which a complementary molecule or amplicon is generated containing a biotinylated unnatural nucleobase, is considered to be encompassed by the phrase “measuring the amount of the unnatural nucleotide in the cDNA using a binding partner that recognizes an unnatural nucleotide” and the like.

In some embodiments, measuring the amount of the unnatural nucleotide in the cDNA using a binding partner that recognizes an unnatural nucleobase comprises a biotin shift assay. A biotin shift assay encompasses any assay that distinguishes biotinylated from unbiotinylated products on the basis of differential mobility binding or not binding to a biotin-binding agent such as streptavidin. The mobility may be, for example, electrophoretic mobility (e.g., gel electrophoretic mobility or capillary electrophoretic mobility) or chromatographic mobility (e.g., using gel filtration, ion exchange, or hydrophobic interaction chromatography).

Where the cDNA was produced from an RNA transcribed from a DNA molecule, the transcription may be in vitro or in vivo. In some embodiments, the transcription is in a bacterium or prokaryote, such as E. coli. In some embodiments, the DNA molecule from which the RNA is transcribed is an ssDNA or dsDNA.

In some embodiments, the method comprises calculating transcription-reverse transcription (T-RT) fidelity (the overall fidelity of transcription and reverse transcription steps). For example, T-RT fidelity can be determined as a ratio of (a) the proportion of cDNA that contains unnatural nucleotide to (b) the proportion of DNA before transcription that contains the unnatural nucleotide. Where a further synthesis step such as an amplification is used to prepare biotinylated DNA, the ratio can be adjusted by a factor to compensate for unnatural base pair loss in the further synthesis step. As shown in the examples, 1.06 is an exemplary value for the factor.

### Methods of Screening RNA Aptamer Candidates

Also disclosed herein are methods of screening RNA aptamer candidates. In some embodiments, the methods comprise incubating a plurality of different RNA oligonucleotides (a “library”) with a target, wherein the RNA oligonucleotides comprise at least one unnatural nucleotide. In some embodiments, the methods comprise performing at least one round of selection for RNA oligonucleotides of the plurality that bind to the target. In some embodiments, the methods comprise isolating enriched RNA oligonucleotides that bind to the target, wherein the isolated enriched RNA oligonucleotides comprise RNA aptamers. In some embodiments, the methods comprise reverse transcribing one or more of the RNA aptamers into cDNAs, wherein the cDNAs comprise an unnatural deoxyribonucleotide at the position complementary to the unnatural nucleobase in the RNA aptamer, thereby providing a library of cDNA molecules corresponding to the RNA aptamers.

In some embodiments, the plurality of different RNA oligonucleotides comprise a randomized nucleotide region. This can be generated, e.g., using mixed pools of nucleotides in certain cycles of a nucleotide synthesis procedure or by performing mutagenic PCR before transcribing oligonucleotides from DNA templates. The randomized nucleotide region may comprise one or a plurality of randomized positions. Where there is a plurality of randomized positions, they may be consecutive or interrupted by one or more nonrandomized nucleotides or segments of nonrandomized nucleotides. In some embodiments, the unnatural nucleobase is within the randomized region (e.g., 3′ to a first randomized position and 5′ to a second randomized position). In some embodiments, the unnatural nucleobase is within 5 or 10 nucleotides of at least one randomized position. In some embodiments, the unnatural nucleobase is immediately adjacent to a randomized position, or is immediately adjacent to two randomized positions.

In some embodiments, the RNA oligonucleotides comprise barcode sequences and/or primer binding sequences. As illustrated in Example 7, barcode sequences can be used to identify the position of the unnatural nucleobase, and primer binding sequences can be used for downstream analysis of active sequences following selection.

In some embodiments, cDNAs produced from the RNA aptamers are sequenced. In some embodiments, cDNAs produced from the RNA aptamers are mutated to generate a plurality of additional sequences, which can then be transcribed into RNA to perform at least one further round of selection. Mutating the cDNAs can be performed, e.g., by error-prone PCR.

In some embodiments, the selection comprises a wash step to remove unbound or weakly bound RNA oligonucleotides. A series of wash steps may be employed where stringency increases, e.g., to provide more selection pressure as the method proceeds.

RNA aptamers identified by the method may be analyzed, e.g., individually, for their ability to bind, agonize, or antagonize the target. In some embodiments, analyzing the RNA aptamers for their ability to bind the target comprises determining a K_{d}, kₒₙ, or k_{off}. In some embodiments, analyzing the RNA aptamers for their ability to agonize the target comprises determining an EC₅₀ value. In some embodiments, analyzing the RNA aptamers for their ability to antagonize the target comprises determining a Kᵢ or IC₅₀ value.

### Additional Features of Polynucleotides

The features described herein may be combined with any disclosed embodiment to the extent feasible. In some embodiments, a polynucleotide comprising an unnatural ribonucleotide comprises at least 15 nucleotides. In some embodiments, the polynucleotide comprises at least 20, 25, 30, 35, 40, 50, 60, 70, 80, 90, or 100 nucleotides. In some embodiments, a polynucleotide comprising an unnatural ribonucleotide comprises one or more ORFs. An ORF may be from any suitable source, sometimes from genomic DNA, mRNA, reverse transcribed RNA or complementary DNA (cDNA) or a nucleic acid library comprising one or more of the foregoing and is from any organism species that contains a nucleic acid sequence of interest, protein of interest, or activity of interest. Non-limiting examples of organisms from which an ORF can be obtained include bacteria, yeast, fungi, human, insect, nematode, bovine, equine, canine, feline, rat or mouse, for example. In some embodiments, a nucleotide and/or nucleic acid reagent or other reagent described herein is isolated or purified. ORFs may be created that include unnatural nucleotides via published in vitro methods. In some cases, a nucleotide or nucleic acid reagent comprises an unnatural nucleobase.

A polynucleotide sometimes comprises a nucleotide sequence adjacent to an ORF that is translated in conjunction with the ORF and encodes an amino acid tag. The tag-encoding nucleotide sequence is located 3′ and/or 5′ of an ORF in the nucleic acid reagent, thereby encoding a tag at the C-terminus or N-terminus of the protein or peptide encoded by the ORF. Any tag that does not abrogate in vitro transcription and/or translation may be utilized and may be appropriately selected by the artisan. Tags may facilitate isolation and/or purification of the desired ORF product from culture or fermentation media. In some instances, libraries of nucleic acid reagents are used with the methods and compositions described herein. For example, a library of at least 100, 1000, 2000, 5000, 10,000, or more than 50,000 unique polynucleotides are present in a library, wherein each polynucleotide comprises at least one unnatural nucleobase.

A polynucleotide can comprise certain elements, e.g., regulatory elements, often selected according to the intended use of the nucleic acid. Any of the following elements can be included in or excluded from a nucleic acid reagent. A polynucleotide, for example, may include one or more or all of the following nucleotide elements: one or more promoter elements, one or more 5′ untranslated regions (5′UTRs), one or more regions into which a target nucleotide sequence may be inserted (an “insertion element”), one or more target nucleotide sequences, one or more 3′ untranslated regions (3′UTRs), and one or more selection elements. A polynucleotide can be provided with one or more of such elements and other elements may be inserted into the nucleic acid before the nucleic acid is introduced into the desired organism. In some embodiments, a provided nucleic acid reagent comprises a promoter, a 5′UTR, an optional 3′UTR and insertion element(s) by which a target nucleotide sequence is inserted (i.e., cloned) into the nucleotide acid reagent. In certain embodiments, a provided nucleic acid reagent comprises a promoter, insertion element(s) and optional 3′UTR, and a 5′ UTR/target nucleotide sequence is inserted with an optional 3′UTR. The elements can be arranged in any order suitable for expression in the chosen expression system (e.g., expression in a chosen organism, or expression in a cell-free system, for example), and in some embodiments a nucleic acid reagent comprises the following elements in the 5′ to 3′ direction: (1) promoter element, 5′UTR, and insertion element(s); (2) promoter element, 5′UTR, and target nucleotide sequence; (3) promoter element, 5′UTR, insertion element(s) and 3′UTR; and (4) promoter element, 5′UTR, target nucleotide sequence and 3′UTR. In some embodiments, the UTR can be optimized to alter or increase transcription or translation of the ORF that are either fully natural or that contain unnatural nucleotides.

Polynucleotides, e.g., expression cassettes and/or expression vectors, can include a variety of regulatory elements, including promoters, enhancers, translational initiation sequences, transcription termination sequences and other elements. A “promoter” is generally a sequence or sequences of DNA that function when in a relatively fixed location in regard to the transcription start site. For example, the promoter can be upstream of the nucleotide triphosphate transporter nucleic acid segment. A “promoter” contains core elements required for basic interaction of RNA polymerase and transcription factors and can contain upstream elements and response elements. “Enhancer” generally refers to a sequence of DNA that functions at no fixed distance from the transcription start site and can be either 5′ or 3″ to the transcription unit. Furthermore, enhancers can be within an intron as well as within the coding sequence itself. They are usually between 10 and 300 nucleotides in length, and they can function in cis. Enhancers function to increase transcription from nearby promoters. Enhancers, like promoters, also often contain response elements that mediate the regulation of transcription. Enhancers often determine the regulation of expression and can be used to alter or optimize ORF expression, including ORFs that are fully natural or that contain unnatural nucleotides.

As noted above, a polynucleotide may also comprise one or more 5′ UTR's, and one or more 3′UTR's. For example, expression vectors used in eukaryotic host cells (e.g., yeast, fungi, insect, plant, animal, human or nucleated cells) and prokaryotic host cells (e.g., virus, bacterium) can contain sequences that signal for the termination of transcription which can affect mRNA expression. These regions can be transcribed as polyadenylated segments in the untranslated portion of the mRNA encoding tissue factor protein. The 3′ untranslated regions also include transcription termination sites. In some preferred embodiments, a transcription unit comprises a polyadenylation region. One benefit of this region is that it increases the likelihood that the transcribed unit will be processed and transported like mRNA. The identification and use of polyadenylation signals in expression constructs is well established. In some preferred embodiments, homologous polyadenylation signals can be used in the transgene constructs.

A 5′ UTR may comprise one or more elements endogenous to the nucleotide sequence from which it originates, and sometimes includes one or more exogenous elements. A 5′ UTR can originate from any suitable nucleic acid, such as genomic DNA, plasmid DNA, RNA or mRNA, for example, from any suitable organism (e.g., virus, bacterium, yeast, fungi, plant, insect or mammal). The artisan may select appropriate elements for the 5′ UTR based upon the chosen expression system (e.g., expression in a chosen organism, or expression in a cell-free system, for example). A 5′ UTR sometimes comprises one or more of the following elements known to the artisan: enhancer sequences (e.g., transcriptional or translational), transcription initiation site, transcription factor binding site, translation regulation site, translation initiation site, translation factor binding site, accessory protein binding site, feedback regulation agent binding sites, Pribnow box, TATA box, -35 element, E-box (helix-loop-helix binding element), ribosome binding site, replicon, internal ribosome entry site (IRES), silencer element and the like. In some embodiments, a promoter element may be isolated such that all 5′ UTR elements necessary for proper conditional regulation are contained in the promoter element fragment, or within a functional subsequence of a promoter element fragment.

A 5′ UTR in the polynucleotide can comprise a translational enhancer nucleotide sequence. A translational enhancer nucleotide sequence often is located between the promoter and the target nucleotide sequence in a polynucleotide. A translational enhancer sequence often binds to a ribosome, sometimes is an 18S rRNA-binding ribonucleotide sequence (i.e., a 40S ribosome binding sequence) and sometimes is an internal ribosome entry sequence (IRES). An IRES generally forms an RNA scaffold with precisely placed RNA tertiary structures that contact a 40S ribosomal subunit via a number of specific intermolecular interactions. Examples of ribosomal enhancer sequences are known and can be identified by the artisan (e.g., Mignone et al., Nucleic Acids Research 33: D141-D146 (2005); Paulous et al., Nucleic Acids Research 31: 722-733 (2003); Akbergenov et al., Nucleic Acids Research 32: 239-247 (2004); Mignone et al., Genome Biology 3(3): reviews0004.1-0001.10 (2002); Gallie, Nucleic Acids Research 30: 3401-3411 (2002); Shaloiko et al., DOI: 10.1002/bit.20267; and Gallie et al., Nucleic Acids Research 15: 3257-3273 (1987)).

A translational enhancer sequence sometimes is a eukaryotic sequence, such as a Kozak consensus sequence or other sequence (e.g., hydroid polyp sequence, GenBank accession no. U07128). A translational enhancer sequence sometimes is a prokaryotic sequence, such as a Shine-Dalgarno consensus sequence. In certain embodiments, the translational enhancer sequence is a viral nucleotide sequence. A translational enhancer sequence sometimes is from a 5′ UTR of a plant virus, such as Tobacco Mosaic Virus (TMV), Alfalfa Mosaic Virus (AMV); Tobacco Etch Virus (ETV); Potato Virus Y (PVY); Turnip Mosaic (poty) Virus and Pea Seed Borne Mosaic Virus, for example. In certain embodiments, an omega sequence about 67 bases in length from TMV is included in the polynucleotide as a translational enhancer sequence (e.g., devoid of guanosine nucleotides and includes a 25-nucleotide long poly (CAA) central region).

A 3′ UTR may comprise one or more elements endogenous to the nucleotide sequence from which it originates and sometimes includes one or more exogenous elements. A 3′ UTR may originate from any suitable nucleic acid, such as genomic DNA, plasmid DNA, RNA or mRNA, for example, from any suitable organism (e.g., a virus, bacterium, yeast, fungi, plant, insect or mammal). The artisan can select appropriate elements for the 3′ UTR based upon the chosen expression system (e.g., expression in a chosen organism, for example). A 3′ UTR sometimes comprises one or more of the following elements known to the artisan: transcription regulation site, transcription initiation site, transcription termination site, transcription factor binding site, translation regulation site, translation termination site, translation initiation site, translation factor binding site, ribosome binding site, replicon, enhancer element, silencer element and polyadenosine tail. A 3′ UTR often includes a polyadenosine tail and sometimes does not, and if a polyadenosine tail is present, one or more adenosine moieties may be added or deleted from it (e.g., about 5, about 10, about 15, about 20, about 25, about 30, about 35, about 40, about 45 or about 50 adenosine moieties may be added or subtracted).

In some embodiments, modification of a 5′ UTR and/or a 3′ UTR is used to alter (e.g., increase, add, decrease or substantially eliminate) the activity of a promoter. Alteration of the promoter activity can in turn alter the activity of a peptide, polypeptide or protein (e.g., enzyme activity for example), by a change in transcription of the nucleotide sequence(s) of interest from an operably linked promoter element comprising the modified 5′ or 3′ UTR. For example, a microorganism can be engineered by genetic modification to express a polynucleotide comprising a modified 5′ or 3′ UTR that can add a novel activity (e.g., an activity not normally found in the host organism) or increase the expression of an existing activity by increasing transcription from a homologous or heterologous promoter operably linked to a nucleotide sequence of interest (e.g., homologous or heterologous nucleotide sequence of interest), in certain embodiments. In some embodiments, a microorganism can be engineered by genetic modification to express a nucleic acid reagent comprising a modified 5′ or 3′ UTR that can decrease the expression of an activity by decreasing or substantially eliminating transcription from a homologous or heterologous promoter operably linked to a nucleotide sequence of interest, in certain embodiments.

### Kits and Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

In some embodiments, a kit includes a suitable packaging material to house the contents of the kit. In some cases, the packaging material is constructed by well-known methods, preferably to provide a sterile, contaminant-free environment. The packaging materials employed herein can include, for example, those customarily utilized in commercial kits sold for use with nucleic acid sequencing systems. Exemplary packaging materials include, without limitation, glass, plastic, paper, foil, and the like, capable of holding within fixed limits a component set forth herein.

The packaging material can include a label which indicates a particular use for the components. The use for the kit that is indicated by the label can be one or more of the methods set forth herein as appropriate for the particular combination of components present in the kit. For example, a label can indicate that the kit is useful for a method of synthesizing a polynucleotide or for a method of determining the sequence of a nucleic acid.

Instructions for use of the packaged reagents or components can also be included in a kit. The instructions will typically include a tangible expression describing reaction parameters, such as the relative amounts of kit components and sample to be admixed, maintenance time periods for reagent/sample admixtures, temperature, buffer conditions, and the like.

It will be understood that not all components necessary for a particular reaction need be present in a particular kit. Rather one or more additional components can be provided from other sources. The instructions provided with a kit can identify the additional component(s) that are to be provided and where they can be obtained.

In some embodiments, a kit is provided that is useful for stably incorporating an unnatural nucleic acid into a cellular nucleic acid, e.g., using the methods provided by the present disclosure

for preparing genetically engineered cells. In one embodiment, a kit described herein includes a genetically engineered cell and one or more unnatural nucleic acids.

In additional embodiments, the kit described herein provides a cell and a nucleic acid molecule containing a heterologous gene for introduction into the cell to thereby provide a genetically engineered cell, such as expression vectors comprising the nucleic acid of any of the embodiments hereinabove described in this paragraph.

### EXAMPLES

### Materials, Methods, and Experimental Procedures for In Vitro and In Vivo Transcription and Reverse Transcription Experiments

The following experimental procedures were used wherever applicable in Examples 1 through 5.

Materials. A complete list of plasmids and primers used in this work is provided in Tables 4 and 5. Primers and natural oligonucleotides were purchased from IDT (Coralville, Iowa). Sequencing was performed by Genewiz (San Diego, CA). Plasmids were purified using a commercial miniprep kit (D4013, Zymo Research; Irvine, CA). PCR products were purified using a commercial DNA purification kit (D4054, Zymo Research) and quantified by A260/A280 absorption using an Infinite M200 Pro plate reader (TECAN). All experiments involving RNA species were done with RNase-free reagents, pipette tips, tubes and gloves to avoid contamination.

Nucleosides of dNaM, dTPT3, NAM, TPT3, d5SICS and dMMO2^{bio} were synthesized (WuXi AppTec; Shanghai, China) and triphosphorylated (TriLink BioTechnologies LLC; San Diego, CA and MyChem LLC; San Diego, CA) commercially. All unnatural oligonucleotides were synthesized and HPLC purified by Biosearch Technologies (Petaluma, CA). All DNA samples containing the unnatural base pair were stored at −20° C. All RNA samples were stored at −80° C.

TABLE 4
Primers. Table 4 discloses SEQ ID NOS 1-12,
respectively, in order of appearance.

| Primer | Sequence |
|---|---|
| AZ01 | GACAAATTAATACGACTCACTATAGGAAACCTGATCATG |
| | TAGATCGAAC |
| AZ38 | CCCCAGGCTTTACACTTTATG |
| AZ67 | TmGGCGGAAACCCCGGGAATCTAACCCGGCTGAACGGAT |
| | T |
| AZ172 | GGAATCTAACCCGGCTGAAC |
| AZ188 | GGAATCTAACCCGGCTGAACCCTCGATGTTGTGGGGGAT |
| | C |
| AZ189 | GATTCCATTCTTTTGTTTGTCTGCTGGCGGAAACCCCGG |
| | GAATC |
| AZ200 | GGAATCTAACCCGGCTGAACGATTCCATTCTTTTGTTTG |
| | TCTGC |
| YZ73 | ATGGGTCTCACACAAACTCGAGTACAACTTTAACTCACA |
| | C |
| YZ74 | ATGGGTCTCGATTCCATTCTTTTGTTTGTCTGC |
| YZ435 | ATGGGTCTCGAAACCTGATCATGTAGATCGAACGG |
| YZ436 | ATGGGTCTCATCTAACCCGGCTGAACGG |
| ED101 | TAATACGACTCACTATAGG |

TABLE 5
Oligonucleotides. Table 5 discloses SEQ
IDNOS 13-34, respectively, in order of
appearance.

| Oligonu- | |
|---|---|
| cleotides | Sequence |
| GFP_Y151_ | CTCGAGTACAACTTTAACTCACACAA |
| TAC | TGTATACATCACGGCAGACAAACAAA |
| | AGAATGGAATC |
| GFP_Y151_ | CTCGAGTACAACTTTAACTCACACAA |
| TAG | TGTAGTAATCACGGCAGACAAACAAA |
| | AGAATGGAATC |
| GFP_Y151_ | CTCGAGTACAACTTTAACTCACACAA |
| AXC | TGTAAXCATCACGGCAGACAAACAAA |
| | AGAATGGAATC |
| GFP_Y151_ | CTCGAGTACAACTTTAACTCACACAA |
| AYC | TGTAAYCATCACGGCAGACAAACAAA |
| | AGAATGGAATC |
| GFP_Y151_ | CTCGAGTACAACTTTAACTCACACAA |
| GXC | TGTAGXCATCACGGCAGACAAACAAA |
| | AGAATGGAATC |
| GFP_Y151_ | CTCGAGTACAACTTTAACTCACACAA |
| GYC | TGTAGYCATCACGGCAGACAAACAAA |
| | AGAATGGAATC |
| GFP_Y151_ | CTCGAGTACAACTTTAACTCACACAA |
| GXT | TGTAGXTATCACGGCAGACAAACAAA |
| | AGAATGGAATC |
| GFP_Y151_ | CTCGAGTACAACTTTAACTCACACAA |
| GYT | TGTAGYTATCACGGCAGACAAACAAA |
| | AGAATGGAATC |
| GFP_Y151_ | CTCGAGTACAACTTTAACTCACACAA |
| AXA | TGTAAXAATCACGGCAGACAAACAAA |
| | AGAATGGAATC |
| GFP_Y151_ | CTCGAGTACAACTTTAACTCACACAA |
| AXT | TGTAAXTATCACGGCAGACAAACAAA |
| | AGAATGGAATC |
| GFP_Y151_ | CTCGAGTACAACTTTAACTCACACAA |
| TXA | TGTATXAATCACGGCAGACAAACAAA |
| | AGAATGGAATC |
| GFP_Y151_ | CTCGAGTACAACTTTAACTCACACAA |
| TXT | TGTATXTATCACGGCAGACAAACAAA |
| | AGAATGGAATC |
| GFP_Y151_ | CTCGAGTACAACTTTAACTCACACAA |
| GXA | TGTAGXAATCACGGCAGACAAACAAA |
| | AGAATGGAATC |
| Mm_tRNA_ | CCTGATCATGTAGATCGAACGGACT |
| GTA | GTAAATCCGTTCAGCCGGGTTAGATTC |
| Mm_tRNA_ | CCTGATCATGTAGATCGAACGGACT |
| CTA | CTAAATCCGTTCAGCCGGGTTAGATTC |
| Mm_tRNA_ | CCTGATCATGTAGATCGAACGGACT |
| GYT | GYTAATCCGTTCAGCCGGGTTAGATTC |
| Mm_tRNA_ | CCTGATCATGTAGATCGAACGGACT |
| GXT | GXTAATCCGTTCAGCCGGGTTAGATTC |
| Mm_tRNA_ | CCTGATCATGTAGATCGAACGGACT |
| GYC | GYCAATCCGTTCAGCCGGGTTAGATTC |
| Mm_tRNA_ | CCTGATCATGTAGATCGAACGGACT |
| GXC | GXCAATCCGTTCAGCCGGGTTAGATTC |
| Mm_tRNA_ | CCTGATCATGTAGATCGAACGGAC |
| AYC | TAYCAATCCGTTCAGCCGGGTTAGATTC |
| Mm_tRNA_ | CCTGATCATGTAGATCGAACGGAC |
| AXC | TAXCAATCCGTTCAGCCGGGTTAGATTC |
| Mm_tRNA_ | CCTGATCATGTAGATCGAACGGACT |
| TYC | TYCAATCCGTTCAGCCGGGTTAGATTC |

PCR reactions with unnatural base pairs. Briefly, the manufacturer's instructions for OneTaq were followed (OneTaq DNA Polymerase, M0480L, New England Biolabs, (NEB)) with the addition of 100 nM dNaMTP and dTPT3TP each. The extension step was adjusted to 4 min in all cases.

Construction of EGFP and tRNA templates. The EGFP template plasmids, pUCCS2_EGFP(NNN) and pUCCYBA_EGFP(NNN), were made by Golden Gate assembly with an EGFP sequence context. The inserts used in all Golden Gate assemblies were PCR products generated with synthesized dNaM-containing oligonucleotides and primers YZ73 and YZ74 (Table 6). Plasmids pUCCS2_EGFP(NNN) and pUCCYBA_EGFP(NNN) were purified after Golden Gate assembly and quantified using Qubit (ThermoFisher). EGFP template plasmids (2 ng) were used in the template-generating PCR reaction with primers ED101 and AZ38 for pUCCS2_EGFP(NNN), and primers ED101 and AZ87 for pUCCYBA_EGFP(NNN). The PCR products were subjected to DpnI digestion and then purified to yield EGFP templates for in vitro transcription.

TABLE 6
Primer Usage

| | RT reaction | Bio-primer | cDNA biotin |
|---|---|---|---|
| Target gene | primer | PCR primer | shift primer |
| EGFP | AZ188 | bio-YZ73/YZ74 | YZ73/AZ172 |
| sfGFP | AZ200 | bio-YZ73/YZ74 | YZ73/AZ172 |
| mazei tRNA | AZ189 | bio-YZ435/YZ546 | YZ435/YZ74 |
| in vivo | | | |

tRNA templates were made by direct PCR from synthesized dNaM-containing oligonucleotides with primers AZO1 and AZ67. The PCR products were purified to yield tRNA templates for in vitro transcription.

The pSyn_sfGFP(NNN)_mm(NNN) plasmids used in SSO in vivo translation experiments were made by Golden Gate assembly. The inserts used in all Golden Gate assemblies were PCR products generated with synthesized dNaM-containing oligonucleotides either with primer set YZ73/YZ74 for mRNA codon insert or primer set YZ435/YZ436 for tRNA anticodon insert. Plasmids pSyn_sfGFP(NNN)_mm(NNN) was purified after Golden Gate assembly and quantified using Qubit.

Biotin shift assay. The retention of the unnatural base pair in templates of RNA species were assayed using d5SICSTP and dMMO2bio-TP with a corresponding primer set. Band intensities were quantified using Image Lab (Bio-Rad). Unnatural base pair retention was normalized by dividing the percentage raw shift of each sample by the percentage raw shift of the synthesized dNaM-containing oligonucleotide template used in the Golden Gate assembly when constructing the EGFP plasmid. Biotin shift assays are discussed in detail in Malyshev et al., A Semi-Synthetic Organism with an Expanded Genetic Alphabet. Nature 2014, 509, 385-388.

In vitro transcription of EGFP mRNAs. Templates (500-1000 ng) were used in each in vitro transcription reaction (HiScribe T7 ARCA with Tailing, E2060S, New England Biolabs, (NEB)) with or without 1.25 mM unnatural ribonucleotriphosphate accordingly, followed by purification (D7010, Zymo Research). The mRNA products were quantified by Qubit and then stored in 5μg aliquots in solution at −80° C.

In vitro transcription of tRNAs. Templates (500-1000 ng) were used in each in vitro transcription reaction (T7 RNA Polymerase, E0251L, NEB) with or without 2 mM unnatural ribonucleoside triphosphate accordingly, followed by purification (D7010, Zymo). The tRNA products were quantified by Qubit and then subjected to refolding (95° C. for 1 min, 37° C. for 1 min, 10° C. for 2 min). All tRNAs were stored in 1800 ng aliquots −80° C.

Reverse transcription. The reverse transcription reactions were conducted according to the manufacturer's instructions of each reverse transcriptase with the following modifications. In all reverse transcription reactions, 1 μg mRNA or 20 ng tRNA, 0.5 mM dNTP and 0.2 mM dNaMTP or dTPT3TP per 20 μL reaction were used unless stated otherwise. For SuperScript III (18080044, ThermoFisher), reactions were incubated at 55° C. for 45 min, inactivated at 70° C. for 15 min, followed by RNase H (M0297S, New England Biolabs, (NEB)) and RNase A (R1253, ThermoFisher) digestion. For SuperScript IV (18090010, ThermoFisher), reactions were incubated at 55° C. for 20 min, inactivated at 80° C. for 10 min, followed by RNase H, RNase A, and Proteinase K (P8107S, New England Biolabs, (NEB)) digestion. For AMV reverse transcriptase (M0277S, New England Biolabs, (NEB)), reactions were incubated at 42° C. for 60 min, inactivated at 80° C. for 5 min, followed by RNase H and RNase A digestion. After digestion, 10 μL of each reaction mixture was denatured with RNA loading dye (B0363S, New England Biolabs, (NEB)) and subjected to 10% denaturing polyacrylamide gel electrophoresis with 8 M urea (CAS 57-13-6, Sigma Aldrich) for cDNA detection. The other 10 μL of the reaction mixture was purified using a commercial RNA purification kit (D7011, Zymo Research; Irvine, CA) and the product cDNA was quantified using Qubit.

Single-strand DNA isolation. The asDNA was prepared via PCR amplification with a biotinylated 5′ primer from the dsDNA template used for the IVT reaction. The product biotinylated dsDNA (bio-dsDNA) was subjected to affinity single-strand isolation protocol using Dynabeads™ MyOne™ Streptavidin C1 (65001, ThermoFisher) according to the manufacturer instruction. Briefly, beads (20 μL) were pre-washed 3 times with WB buffer and then mixed with purified bio-dsDNA (20 μL, ˜50 ng/μL). The mixture was incubated for 2 h at 37° C. with gentle shaking. The beads were separated from the buffer using a magnetic stand. The beads were then washed 3 times with WB buffer, and the unbiotinylated strand was eluted using 100 μL 0.1 M NaOH (wash time <30 s). The eluted unbiotinylated asDNA was then purified using column purification.

SSO in vivo translation. A 2 mL overnight culture of YZ3+pGEX-MbPylRS TetR cells in 2×YT (Y2377, Sigma Aldrich) supplemented with 50 mM potassium phosphate (CAS 7778-77-0, Sigma Aldrich), 5 μg/mL chloramphenicol (CAS 56-75-7, Sigma Aldrich) and 100 μg/mL carbenicillin (C1613, Sigma Aldrich) (herein afterward referred to in this section as “media”) was diluted to an OD600 of 0.03 in the same media, and grown to an OD600 of 0.3 to 0.4. The culture was rapidly cooled in an ice water bath for 5 min with shaking, and then pelleted at 3,200×g for 10 min. Cells were next washed twice with one culture volume of pre-chilled autoclaved Milli-Q H2O. Cells were then resuspended in additional chilled H₂O, to an OD600 of 50-60. For each sample tested, 50 μL of the resulting electrocompetent cells were combined with 0.5 ng of Golden Gate assembled plasmid containing the UBP embedded within the sfGFP and tRNA^{Pyl} genes and then transferred to a pre-chilled electroporation cuvette (0.2 cm gap). Cells were electroporated (Gene Pulser II; Bio-Rad) according to the manufacturer's instructions for bacteria (25 kV, 2.5 μF, and 200Ω resistor), then immediately diluted with 950 μL of pre-warmed media. 10 μL of this dilution was then diluted with pre-warmed media to a final volume of 50 μL, supplemented with 150 mM dNaMTP and 10 μM dTPT3TP. The transformation was allowed to recover at 37° C. for 1 h. The recovery culture was plated on solid media supplemented with 50 μg/mL zeocin (R25001, ThermoFisher), 150 μM dNaMTP, 10 μM dTPT3TP, and 2% w/v agar, then allowed to grow at 37° C. overnight.

Single colonies were isolated and used to inoculate 300 μL liquid media supplemented with 50 μg/mL zeocin (herein afterward referred to in this section as “growth media”) and provided 150 μM dNaMTP, and 10 μM dTPT3TP, then monitored for cell growth via OD600 using an Envision 2103 Multilabel Plate Reader (Perkin Elmer) with a 590/20 nm filter. Cells were collected at an OD600 of ˜0.7, and then an aliquot (100 μL) was subjected to miniprep. Isolated plasmids were subjected to biotin shift assay to determine UBP retention. Colonies that were shown to have retained the UBP were then diluted back to an OD600 of ˜0.1-0.2 in 300 μL growth media supplemented with 150 μM dNaMTP, and 10 μM dTPT3TP. At an OD600 of cultures were supplemented with 250 μM NaMTP and 30 μM TPT3TP unless stated otherwise, as well as 10 mM of the ncAA N6-(2-azidoethoxy)-carbonyl-L-lysine (AzK). The culture was then grown for and additional 20 min before adding IPTG (CAS 367-93-1, Sigma Aldrich) to a concentration of 1 mM and grown for 1 h to induce the transcription of the T7 RNA polymerase, the tRNA^{Pyl}, and the PylRS. Cells were monitored for growth (OD600) and GFP fluorescence every 30 min. Expression of sfGFP was then induced with 100 ng/mL anhydrotetracycline (CAS 13803-65-1, Sigma Aldrich). After an additional 3 h of growth, cell cultures were collected and cooled on ice. 50 μL of the culture was used for plasmid isolation to determine UBP retention (biotin shift assay); the remaining 250 μL of the culture was used for total RNA extraction to measure T-RT retention.

Total RNA extraction. Following the in vivo translation experiment, the E. coli culture was collected and centrifuged (Centrifuge 5415 C, Eppendorf) at 10,000 rpm for 30 seconds, and the supernatant was discarded. 1 mL TRIzol (15596026, ThermoFisher) was then added to each sample. The mixture was homogenized and incubated at room temperature for 5 min. 200 μL chloroform (CAS 67-66-3, Sigma Aldrich) was added to each sample and the mixture was vortexed to homogenization, followed by room temperature incubation for 3 min to allow for phase separation. Next, the sample was centrifuged at 12,000 rpm for 15 min at 4° C., the colorless aqueous phase was collected into a new tube and 500 μL isopropyl alcohol (CAS 67-63-0, Sigma Aldrich) was added to the aqueous phase. After incubation at room temperature for min, the sample was centrifuged at 7,000 rpm for 10 min at 4° C. and the supernatant was discarded. The sample was then washed with 2′ 1 mL 75% ethanol. The lids of the tubes were kept open to allow the sample to dry for 30 min at room temperature, and the resulting total RNA was dissolved with 20 μL RNase-free water. The concentration of the total RNA was measured using Qubit.

### Example 1. Sequential In Vitro Transcription (IVT) and Reverse Transcription

To explore the ability of reverse transcriptases to productively recognize RNA containing an UBP, sequential in vitro transcription (IVT) and reverse transcription was performed with the commercially available reverse transcriptases: SuperScript III, SuperScript IV and AMV reverse transcriptase. DNA containing the EGFP gene with dNaM or dTPT3 located at the position encoding the second nucleotide of codon 151 was PCR amplified and used as a template for IVT reactions, which were supplemented with the corresponding unnatural ribonucleoside triphosphate, but otherwise run according to manufacturer instructions. The RNA was purified and then used as a template for RT reactions that were performed with or without unnatural deoxyribonucleoside triphosphate (in addition, the primer installed a 3′-extension to facilitate analysis, see following paragraph). After 1 hour, half of the RT reaction was subjected to PAGE gel electrophoresis to qualitatively assess the presence of full length and truncated products, and the other half was purified for subsequent characterization of the retention of the unnatural nucleotide.

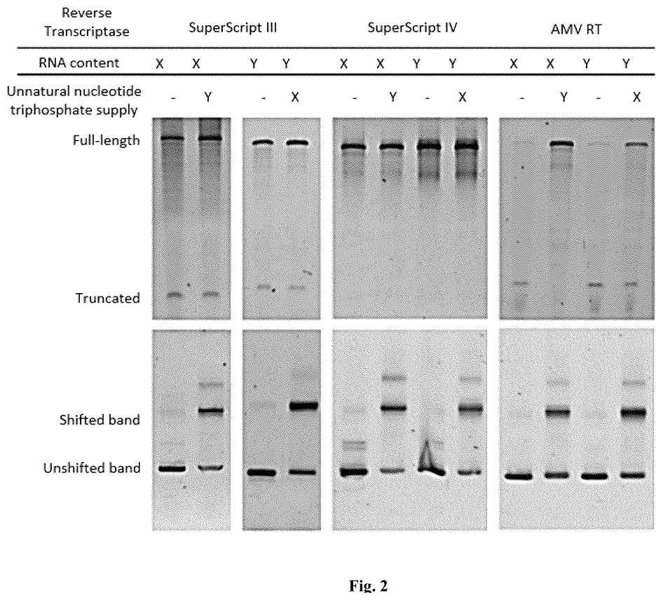

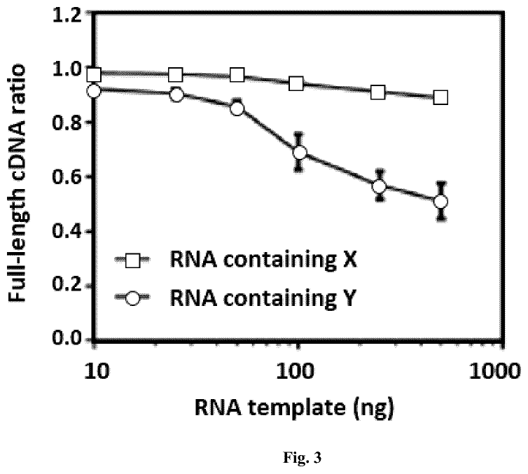

With AMV reverse transcriptase, RNA templates containing either NaM or TPT3 yielded mostly only truncated cDNA product when dTPT3TP or dNaMTP was absent, and mostly only full-length product when dTPT3TP or dNaMTP was provided (FIG. 2). In contrast, with SuperScript III or SuperScript IV, full length cDNA product was observed with either template regardless of whether the unnatural triphosphates were added (FIG. 2). A biotin shift assay , performed essentially as described in Malyshev et al., A Semi-Synthetic Organism with an Expanded Genetic Alphabet. Nature 2014, 509, 385-388, was used to detect the presence of the unnatural nucleotide in the RT product. The purified cDNA was amplified by PCR in the presence of each natural dNTP as well as dMMO2bioTP (a biotinylated analog of dNaMTP) and d5SICSTP (an analog of dTPT3TP that pairs with dMMO2 bio during replication better than dTPT3TP itself). The use of a 3′-primer that anneals to the sequence installed by the RT primer (see above) prevented the amplification of any DNA template remaining from the original IVT reaction (FIG. 3). PCR products were then incubated with streptavidin and subjected to PAGE electrophoresis, where the resulting ratio of shifted to unshifted bands indicates the percentage of the cDNA that contains an unnatural nucleotide. As expected, when unnatural triphosphates were withheld from the RT reaction, no shifted products were observed. In contrast, when the complementary unnatural triphosphate was added to the RT reaction, a substantial shift was observed, indicating that with all three reverse transcriptases, a significant amount of the cDNA product contained the unnatural nucleotide (FIG. 2).

### Example 2. Study of Effect of tRNA Template Concentration

tRNA templates produced by IVT of PCR products from synthetic oligonucleotides containing dNaM or dTPT3 at positions corresponding to the second nucleotide of the anticodon were used to study the effect of tRNA template concentration on efficiency of reverse transcription of unnatural nucleobases. At the highest concentration of tRNA (25 ng/μL), reverse transcription of the NaM or TPT3 templates in the presence of their corresponding unnatural deoxyribotriphosphate resulted in 88% and 44% full-length product, respectively. Interestingly, at lower tRNA template concentrations, the percentage of full-length product increased. With 0.5 μg/mL template, reverse transcription resulted in 97% and 92% full-length product with the NaM or TPT3 templates, respectively (FIG. 3, Table 1).

TABLE 1
Raw data for RNA concentration dependency
of SuperScript III RT reaction full-length
cDNA product ratio using RNA containing NaM or TPT3.

| | RNA containing NaM | | | RNA containing TPT3 | | |
|---|---|---|---|---|---|---|
| RNA | Full-length | | | Full-length | | |
| (ng/ | product ratio | | | product ratio | | |
| reaction) | #1 | #2 | #3 | #1 | #2 | #3 |
| 500 | 0.8789 | 0.9028 | 0.8818 | 0.4423 | 0.5734 | 0.5148 |
| 250 | 0.9008 | 0.9155 | 0.9186 | 0.5226 | 0.5589 | 0.6222 |
| 100 | 0.9247 | 0.9477 | 0.9489 | 0.7157 | 0.6153 | 0.7373 |
| 50 | 0.9567 | 0.9688 | 0.9747 | 0.8543 | 0.8374 | 0.8786 |
| 25 | 0.9651 | 0.9759 | 0.9844 | 0.9061 | 0.9217 | 0.8854 |
| 10 | 0.9731 | 0.9757 | 0.9918 | 0.9167 | 0.9401 | 0.9065 |

### Example 3. Assay for UBP Retention After Sequential In Vitro Transcription (IVT) and Reverse Transcription

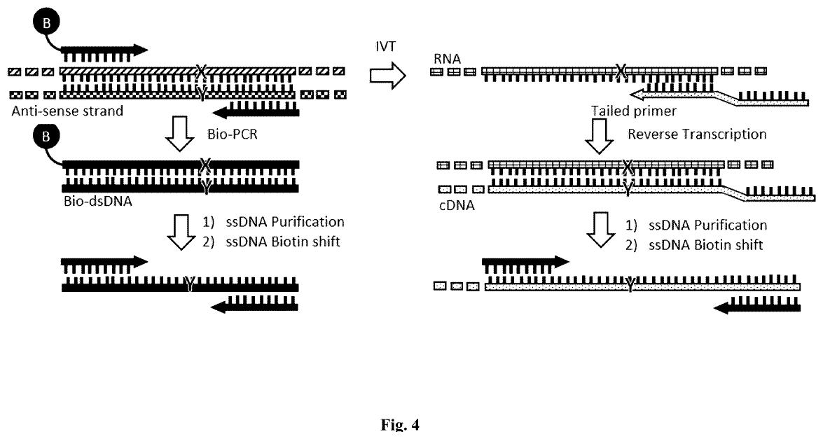

An assay was developed to measure UBP retention quantitatively after sequential in vitro transcription (IVT) with T7 RNA polymerase and reverse transcription (RT) with the commercially available reverse transcriptases: SuperScript III, SuperScript IV and AMV reverse transcriptase. In order to focus on the unnatural nucleotide loss that occurs during IVT and RT only (i.e. to exclude any loss occurring during the PCR preparation of the IVT template), the assay also analyzed the unnatural nucleotide content of the anti-sense DNA template (R(asDNA) (FIG. 4). The combined T-RT fidelty was calculated as:

$$T - RT Retention = α \frac{R ( cDNA )}{R ( asDNA )} ,$$

where the constant, α=1.06, is included to account for the contribution of UBP loss in the additional PCR step required to prepare the bio-dsDNA. As the T-RT retention corresponds to unnatural nucleotide loss during both transcription and reverse transcription, it provides a lower bound of unnatural nucleotide retention during either step of the T-RT reaction.

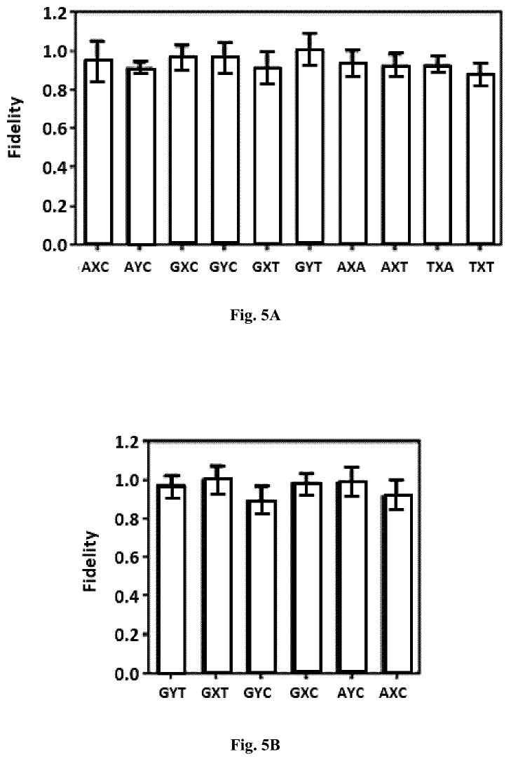

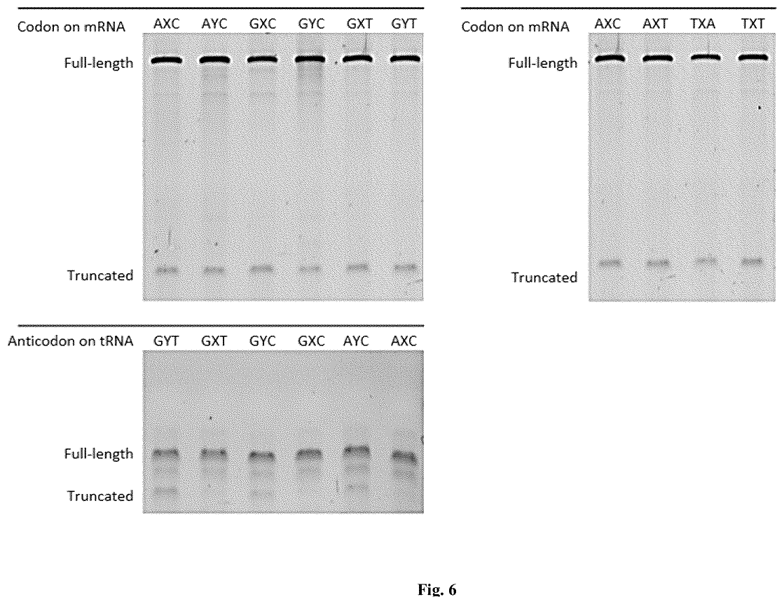

The T-RT fidelity assay was first applied to determine the lower bound of IVT transcription fidelity with EGFP mRNAs containing an unnatural 151st codon, including AXC, AYC, GXC, GYC, GXT, or GYT (X═NaM and Y=TPT3), each of which has been used to express unnatural protein in mammalian cells. Remarkably, all sequences with either NaM or TPT3 produced full-length cDNA as the major product with combined T-RT retentions of 90% to 100% (FIG. 5A, FIG. 6). At least in this sequence context, the unnatural base pair is transcribed (and reverse transcribed) in vitro with reasonable fidelity.

Next, the T-RT of M. mazei tRNA with anticodons GYT, GXT, GYC, GXC, CYA, and CXA was explored. Each tRNA gene, regardless of whether it contained NaM or TPT3, again yielded full-length cDNAs as the major product and with unnatural nucleotide retentions ranging from 90% to 100% (FIG. 5B, FIG. 6). The increased structure of tRNA did not apparently impede its in vitro transcription and reverse transcription with unnatural anticodons.

It was previously reported that HEK293T cells are able to use EGFP(GXC) mRNA and M mazei tRNA(GYC) to produce EGFP protein containing the ncAA AzK. (Zhou et al., Progress toward Eukaryotic Semisynthetic Organisms: Translation of Unnatural Codons. J. Am. Chem. Soc. 2019, 141, 20166-20170.) In those previous experiments, the HEK293T cells were provided with the AzK and transfected with mRNA and tRNA containing unnatural codons and anticodons, respectively, as well as a DNA plasmid encoding the chimeric PylRS which charges the mazei tRNA with AzK. 80% of the DNA template used to prepare the mRNA contained the unnatural nucleotide and 70% of the protein expressed in vivo contained AzK. With the above analysis of the minimum transcription fidelity of the EGFP(GXC) gene, the translation fidelity of the eukaryotic ribosome is estimated as:

$$F ( translation ) = \frac{F ( protein shift )}{F ( replication ) \times F ( transcription )} = 91 \% .$$

Several unnatural codons, including AXA, AXT, TXA, and TXT, have previously been identified in E. coli SSO as well retained during DNA replication but only inefficiently produced protein with an ncAA. (Fischer et al., New Codons for Efficient Production of Unnatural Proteins in a Semisynthetic Organism. Nat. Chem. Biol. 2020, 16, 570-576.) This suggests that they are not well transcribed by T7 RNAP in the SSO and/or that they are not well decoded at the ribosome. DNA individually containing each codon was subjected to the developed in vitro T-RT assay. Each template was again shown to produce full length cDNA as the major product with unnatural nucleotide retentions of approximately 90% (FIG. 5A). This data demonstrates that transcription is relatively efficient and indicates that these codons are unable to efficiently participate in translation.

### Example 4. Characterization of In Vivo Transcription in E. coli SSO

The T-RT retention assay developed in Example 3 was used to characterize RNA isolated from the E. coli SSO. ML2 cells were transformed with the pSyn plasmid encoding the sfGFP gene containing 151st codons AXC, GXC, or GXT and the M. mazei tRNA gene containing the corresponding anticodons GYT, GYC, or AYC, respectively. In each case, the SSO was previously shown to produce unnatural protein with high fidelity (Fischer, E. C., et al., Nat. Chem. Biol.

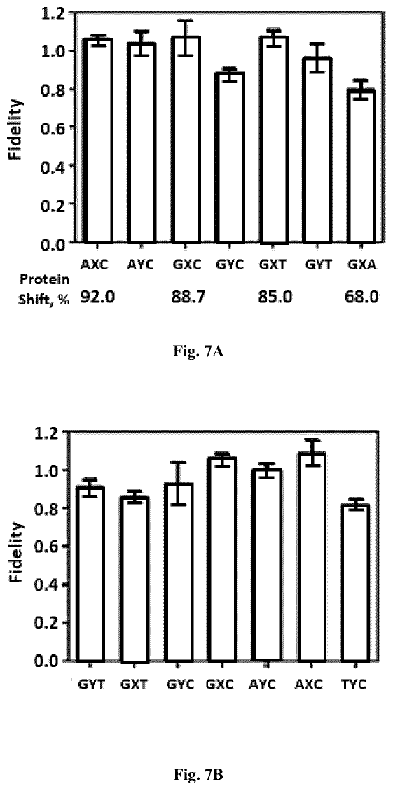

2020, 16, 570-576). Here, the retention of the unnatural nucleotide in the asDNA as well as within each mRNA and tRNA was analyzed as described above. The data revealed that transcription of the NaM codons proceeded in the SSO with virtually no loss of the unnatural nucleotide. For the tRNAs, retention of TPT3 anticodons ranged from 85% to 100% (FIGS. 7A-B, Table 2).

TABLE 2
Raw data of T-RT retention and standard deviation of mRNA and
tRNA extracted from SSO in vivo translation experiments. (n = 3).

| | T-RT retention | Standard deviation |
|---|---|---|
| Codon AXC | 1.06 | 0.02 |
| Codon AYC | 1.04 | 0.06 |
| Codon GXC | 1.07 | 0.09 |
| Codon GYC | 0.88 | 0.03 |
| Codon GXT | 1.07 | 0.04 |
| Codon GYT | 0.96 | 0.07 |
| Codon GXA | 0.80 | 0.05 |
| Anticodon GYT | 0.91 | 0.04 |
| Anticodon GXT | 0.86 | 0.03 |
| Anticodon GYC | 0.93 | 0.11 |
| Anticodon GXC | 1.06 | 0.03 |
| Anticodon AYC | 1.00 | 0.03 |
| Anticodon AXC | 1.09 | 0.07 |
| Anticodon TYC | 0.82 | 0.03 |

The data indicate that the transcription fidelity of mRNA containing NaM is high, and that while the transcription fidelity of tRNA containing TPT3 is somewhat lower, this does not result in reduced fidelity of ncAA incorporation.

In contrast to the codons examined above, E. coli SSO was previously shown to be unable to efficiently produce sfGFP protein using TPT3 codons AYC, GYC, or GYT (again at codon 151 and with the M. mazei tRNA containing the corresponding unnatural anticodons) (Fischer, E. C., et al., Nat. Chem. Biol. 2020, 16, 570-576). Here, the SSO transcription of the corresponding mRNAs and tRNAs was examined (FIGS. 7A-B, Table 2). The data revealed that both mRNA and tRNA containing each of the less functional codon/anticodon pairs are produced with efficiencies and fidelities indistinguishable from the previously analyzed pairs that mediated high level ncAA incorporation. This indicates that the poor performance of the AYC, GYC, or GYT codons in the SSO results from reduced translation efficiency by the E. coli ribosome. That is, in the E. coli SSO, translation is generally more sensitive than transcription to UBP sequence context.

In addition to the TPT3 codons that are not well translated, one NaM codon, GXA, produced sfGFP with a somewhat compromised ncAA incorporation fidelity (50-60%), despite high retention in the DNA. When the RNA produced in the SSO harboring this codon/anticodon pair was examined, both the tRNA, and especially the mRNA, were found to be produced with a somewhat lower fidelity, approximately 80% in both cases (FIGS. 7A-B, Table 2). Given the potential for a non-linear contribution of natural mRNA (due to more efficient translation), this data suggest that in contrast to the other codons, a significant contribution to the reduced ncAA incorporation fidelity of the GXA codon in the SSO arises from a reduced fidelity of transcription.

### Example 5. Impact of Unnatural Ribonucleotide Trisphosphate Concentration on Transcription in SSO

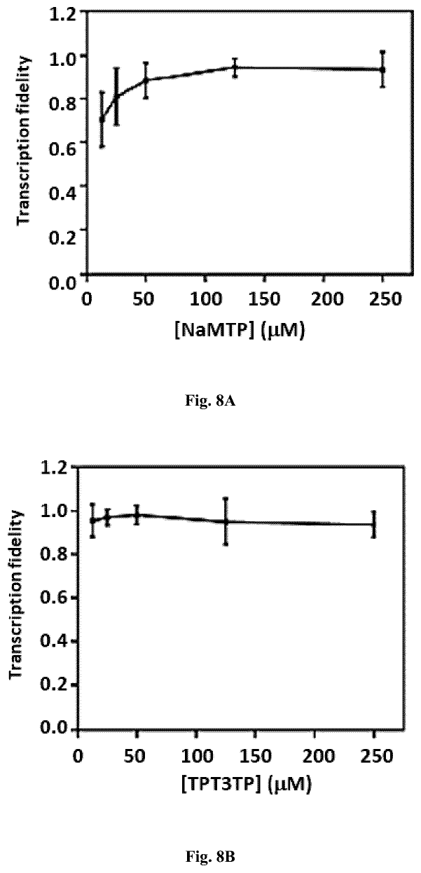

The T-RT fidelity assay described above was further used to explore the explore the dependence of transcription fidelity on unnatural ribonucleotide triphosphate concentration. SSO harboring sfGFP(GXT) and M. mazei tRNA(AYC) was grown as above except that varying amounts of either NaMTP or TPT3TP were provided. When the concentration of TPT3TP was held constant at 250 mM, and the concentration of NaMTP was decreased, retention of NaM in the mRNA remained high until the concentration dropped to less than 50 μM (FIGS. 8A-B, Table 3). When the concentration of NaMTP was held constant at 250 mM, and the concentration of TPT3TP was varied, retention of TPT3 in the tRNA remained high even at the lowest concentration examined (10 μM) (FIGS. 8A-B, Table 3). Thus, the SSO can tolerate lower concentrations of TPT3TP than NaMTP.

TABLE 3
Raw data of T-RT retention′s dependency on either NaMTP or
TPT3TP concentration in SSO in vivo translation experiments. (n = 3).

| | NaMTP concentration (mM) | T-RT retention | Standard deviation |
|---|---|---|---|
| mRNA | 250 | 0.93 | 0.07 |
| | 125 | 0.94 | 0.03 |
| | 50 | 0.88 | 0.07 |
| | 25 | 0.81 | 0.11 |
| | 12.5 | 0.70 | 0.10 |
| | TPT3TP concentration (mM) | T-RT retention | Standard deviation |
| tRNA | 250 | 0.94 | 0.05 |
| | 125 | 0.95 | 0.09 |
| | 50 | 0.98 | 0.04 |
| | 25 | 0.97 | 0.03 |
| | 12.5 | 0.95 | 0.06 |

### Example 7. Enabling the Expansion of RNA Aptamer Selection Using Transcription and Reverse Transcription

To develop RNA aptamers targeting a protein of interest, libraries of RNA are first generated from DNA by IVT, subjected to selection to enrich the library in desired RNAs, converted by RT back into DNA for PCR amplification, and then analyzed or converted back into RNA by IVT and subjected to additional rounds of selection. Thus, to develop RNA aptamers comprising unnatural nucleotides, DNA containing the unnatural nucleotides must be efficiently reverse transcribed into RNA comprising the unnatural nucleotides. In this example, a series of related DNA oligonucleotides with an unnatural nucleotide are converted into RNA with the corresponding unnatural nucleotide, which are then subjected to selection for inhibitory potency. The oligonucleotides may be about 100 bases in length. A region of about 40 nucleotides in an initial DNA oligonucleotide is randomized, and a single dNaM is incorporated at a plurality (e.g., 3) of different positions of the region, flanked by barcode sequences (to identify the unnatural nucleotide position) and primer binding sequences. A plurality (e.g., 3) of related DNA libraries are thus generated. An equimolar mixture of the plurality of randomized oligonucleotide libraries is PCR amplified in reactions that include dTPT3TP and dNaMTP. The primer that primes synthesis of the dTPT3 nucleotide includes a biotin tag attached to its 5′ end via a disulfide, or other cleavable moiety, which are commercially available and commonly used. After amplification, the dsDNA is purified by binding to streptavidin coated magnetic beads, subjecting the beads to buffer washing steps, and then washing with 0.1 mM NaOH to elute the dNaM-containing ssDNA library. The dTPT3-containing ssDNA library can be released from the beads by reductive cleavage using 30 mM Tris(2-carboxyethyl)phosphine (TCEP) (or any other suitable reagent). Either ssDNA library can then be used as template for a T7 RNA polymerase-mediated IVT reaction supplemented with the appropriate unnatural ribotriphosphate (TPT3TP or NaMTP). DNA is degraded nucleolytically and the library is purified (e.g., with a spin column such as the Zymo ssDNA/RNA purification kit).

The library is folded. The resulting folded library is then subjected to selection for binding to the protein of interest. The library is incubated with the target protein of interest, for example immobilized on high-protein adsorption ELISA plates, washed, and then eluted by washing three times with formamide. Selection pressure for binding to the protein of interest is increased through various methods, including by gradually in subsequent rounds of selection raising the concentration of salt in the washing buffer or adding yeast tRNA as a binding competitor in the binding buffer. After each round of selection, the RNAs that bind to the protein of interest are isolated, and the RNA oligonucleotides are eluted. The RNA oligonucleotides are reverse transcribed into cDNA according to methods described herein. The cDNA is PCR amplified with dTPT3TP and dNaMTP and with the same biotinylated primer, and subjected to additional rounds of selection as desired, thereby providing an enriched set of aptamer.

After several rounds of selection following the above steps, the enriched individual RNA aptamers are reverse transcribed into cDNA, PCR amplified, and sequenced (e.g., wherein the unnatural nucleotide is replaced with a natural nucleotide for sequencing, and the barcode sequences are relied upon for identification of the unnatural nucleotide position). Sequence homology among the enriched RNA oligonucleotides is studied, and a subset of sequences are selected for further characterization. Selected RNA aptamers are then synthesized and folded. Each aptamer is then individually analyzed for its ability to bind the target protein (or inhibit its activity if the target protein is an enzyme). The inhibition potency of the aptamers is quantified as K_{d} or Kᵢ values. Optionally, the most promising RNA oligonucleotides can be reverse transcribed into cDNA, and its sequence randomized further via error-prone PCR to generate additional libraries for further rounds of selection.

* * *

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method of reverse transcribing a polynucleotide comprising an unnatural ribonucleotide, comprising reverse transcribing the polynucleotide with a reverse transcriptase in the presence of an unnatural dNTP comprising an unnatural nucleobase, wherein the reverse transcriptase polymerizes a cDNA into which the unnatural dNTP is incorporated as an unnatural nucleotide.

2. The method of claim 1, wherein:
(a) the polynucleotide is present at a concentration less than or equal to about 500 nM;
(b) the reverse transcriptase is SuperScript III;
(c) the unnatural dNTP is not dTPT3TP;
(d) the method further comprises measuring the amount of the unnatural nucleotide in the cDNA using a binding partner that recognizes the unnatural nucleotide;
(e) the reverse transcriptase produces full length cDNA and at least 25% of the full length cDNA comprises the unnatural nucleotide; and/or
(f) the polynucleotide is a tRNA, mRNA, RNA aptamer, or a member of a plurality of RNA aptamer candidates and/or the polynucleotide is an RNA, optionally wherein the RNA is an mRNA or tRNA;
and/or the method further comprises measuring the amount of the unnatural nucleotide in the cDNA.

3. (canceled)

4. (canceled)

5. A method of measuring incorporation of an unnatural nucleotide, comprising:
a. transcribing a polynucleotide comprising an unnatural deoxyribonucleotide with an RNA polymerase in the presence of an unnatural NTP comprising a first unnatural nucleobase to produce an RNA comprising a first unnatural nucleotide;
b. reverse transcribing the RNA with a reverse transcriptase in the presence of an unnatural dNTP comprising a second unnatural nucleobase,
wherein the reverse transcriptase polymerizes a cDNA into which the unnatural NTP is incorporated as a second unnatural nucleotide; and
c. measuring the amount of the second unnatural nucleotide in the cDNA.

6. The method of claim 5, wherein the transcribing step is in vivo.

7. The method of claim 5 the immediately preceding claim, wherein the transcribing step is in a prokaryote or bacterium, optionally wherein the transcribing step is in E. coli.

8. (canceled)

9. (canceled)

10. The method of claim 5, wherein the amount of the second unnatural nucleotide in the cDNA molecule is measured relative to the amount of the unnatural deoxyribonucleotide in the polynucleotide before transcription; and/or wherein the measuring comprises:
a. performing a biotin shift assay on the polynucleotide before transcription to determine the proportion of the polynucleotide before transcription that contains the unnatural nucleotide; and
b. performing a biotin shift assay on the cDNA to determine the proportion of the cDNA that contains containing the unnatural nucleotide; and/or
wherein the amount of the unnatural nucleotide or the second unnatural nucleotide in the cDNA is measured using a binding partner that binds an unnatural nucleobase; and/or
wherein measuring the amount of the unnatural nucleotide or the second unnatural nucleotide in the cDNA comprises a gel shift assay or biotin shift assay.

11. (canceled)

12. (canceled)

13. (canceled)

14. The method of claim 10, wherein the biotin shift assay comprises:
a. amplifying the cDNA in the presence of an unnatural dNTP comprising a biotinylated nucleobase that pairs with the unnatural nucleotide in the cDNA;
b. separating DNA amplification products comprising the biotinylated nucleotide from DNA amplification products not comprising the biotinylated nucleotide; and
c. measuring the amount of DNA amplification products comprising the biotinylated nucleotide and DNA amplification products not comprising the biotinylated nucleotide, or a ratio of DNA amplification products comprising the biotinylated nucleotide to DNA amplification products not comprising the biotinylated nucleotide, or the proportion of cDNA that contains the unnatural nucleotide.

15. (Canceled)

16. (canceled)

17. The method of claim 1, wherein the RNA or polynucleotide is present during reverse transcription at a concentration less than or equal to about 1 μM; and/or
wherein the RNA or polynucleotide is present during reverse transcription at a concentration in the range of about 1-10 nM, about 10-20 nM, about 20-30 nM, about 30-40 nM, about 40-nM, about 50-75 nM, about 75-100 nM, about 100-150 nM, about 150-200 nM, about 200-300 nM, about 300-400 nM, or about 400-500 nM; and/or
wherein the reverse transcriptase produces full length cDNA and wherein at least 25% of the full length cDNA comprises the unnatural nucleotide.

18. (canceled)

19. (canceled)

20. (canceled)

21. The method of claim 1, wherein the RNA or polynucleotide comprising the unnatural ribonucleotide is an mRNA, and wherein:
the unnatural ribonucleotide (X or Y) is located at the first position (X—N—N or Y—N—N) of a codon of the mRNA;
the unnatural ribonucleotide (X or Y) is located at the middle position (N—X—N or N—Y—N) of a codon of the mRNA;
the unnatural ribonucleotide (X or Y) is located at the last position (N—N—X or N—N—Y) of a codon of the mRNA; or
the codon containing the unnatural ribonucleotide in the mRNA is AXC, AYC, GXC, GYC, GXT, GYT, AXA, AXT, TXA, or TXT.

22. (canceled)

23. (canceled)

24. (canceled)

25. (canceled)

26. The method of claim 1, wherein the RNA or polynucleotide comprising the unnatural ribonucleotide is a tRNA, and wherein:
the unnatural ribonucleotide (X or Y) is located at the first position (X—N—N or Y—N—N) of the anticodon of the tRNA,
the unnatural ribonucleotide (X or Y) is located at the middle position (N—X—N or N—Y—N) of the anticodon of the tRNA;
the unnatural ribonucleotide (X or Y) is located at the last position (N—N—X or N—N—Y) of the anticodon of the tRNA, or
the anticodon of the tRNA is GYT, GXT, GYC, GXC, CYA, CXA, AYC, or AXC.

27. (canceled)

28. (canceled)

29. (canceled)

30. (canceled)

31. The method of claim 1, wherein the unnatural ribonucleotide is X, wherein X comprises 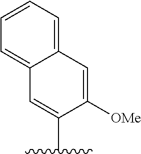 [SMILES: *c1cc2ccccc2cc1OC]
ribonucleotide (NaM); and/or
as the nucleobase of the unnatural
wherein the unnatural ribonucleotide is Y, wherein Y comprises 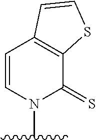 [SMILES: *n1ccc2ccsc2c1=S]
as the nucleobase of the unnatural ribonucleotide (TPT3); and/or
wherein the RNA is an RNA aptamer.

32. (canceled)

33. (canceled)

34. A method of screening RNA aptamer candidates comprising:
a. incubating a plurality of different RNA oligonucleotides with a target, wherein the RNA oligonucleotides comprise at least one unnatural nucleotide;
b. performing at least one round of selection for RNA oligonucleotides of the plurality that bind to the target;
c. isolating enriched RNA oligonucleotides that bind to the target, wherein the isolated enriched RNA oligonucleotides comprise RNA aptamers; and
d. reverse transcribing one or more of the RNA aptamers into cDNAs, wherein the cDNAs comprise an unnatural deoxyribonucleotide at the position complementary to the at least one unnatural nucleotide in the RNA aptamer, thereby providing a library of cDNA molecules corresponding to the RNA aptamers.

35. The method of claim 34, wherein the plurality of different RNA oligonucleotides comprise a randomized nucleotide region; and/or
wherein the RNA oligonucleotides comprise barcode sequences and/or primer binding sequences; and/or
wherein the method further comprises sequencing the cDNA molecules; and/or
wherein performing at least one round of selection comprises a wash step to remove unbound or weakly bound RNA oligonucleotides; and/or
wherein the method further comprises mutating the sequence of the cDNA molecules to generate a plurality of additional sequences; and/or
wherein the method further comprises increasing selection pressure for binding to the target in an additional round of selection.

36. The method of claim 35, wherein the randomized nucleotide region comprises the at least one unnatural nucleotide; and/or
wherein the plurality of additional sequences is transcribed into RNA and subjected to at least one additional round of selection for RNA aptamers that bind to the target, optionally wherein mutating the sequence of the cDNA molecules comprises error-prone PCR; and/or
wherein increasing selection pressure comprises performing one or more washing steps at a higher salt concentration than in a previous round and/or including a binding competitor during the selection.

37. (canceled)

38. (canceled)

39. (canceled)

40. (canceled)

41. (canceled)

42. (canceled)

43. (canceled)

44. (canceled)

45. The method of claim 34, further comprising
analyzing the RNA aptamers for their ability to bind the target; and/or
further comprising analyzing the RNA aptamers for their ability to agonize the target; and/or
further comprising analyzing the RNA aptamers for their ability to antagonize the target.

46. The method of claim 45, wherein
analyzing the RNA aptamers for their ability to bind the target comprises determining a K_{d}, kₒₙ, or k_{off}; and/or
wherein analyzing the RNA aptamers for their ability to agonize the target comprises determining an EC₅₀ value; and/or
wherein analyzing the RNA aptamers for their ability to antagonize the target comprises determining a Kᵢ or IC₅₀ value.

47. (canceled)

48. (canceled)

49. (canceled)

50. (canceled)

51. The method of claim 34, wherein at least one unnatural nucleotide comprises:

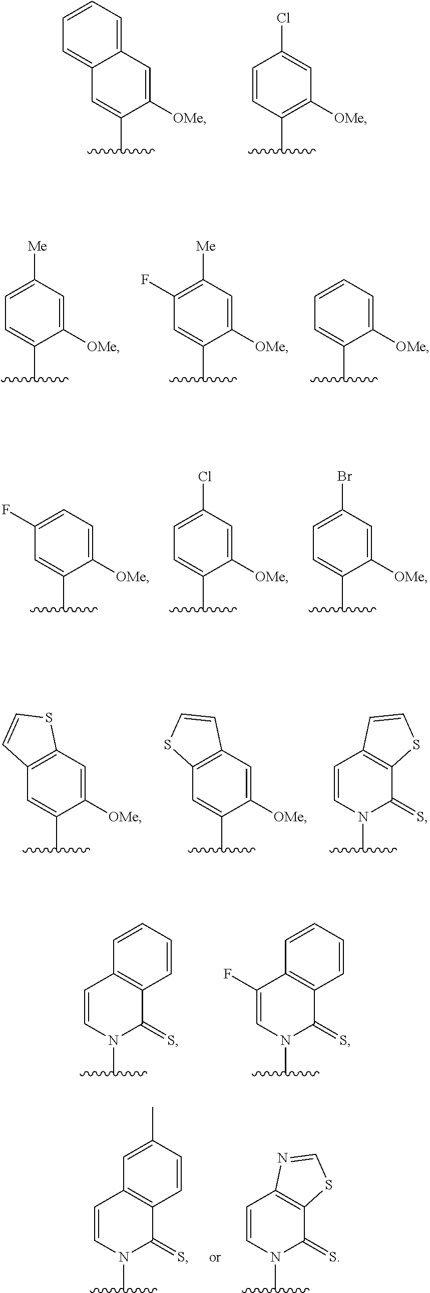

52. The method of claim 51, wherein at least one unnatural nucleotide in a polynucleotide that undergoes reverse transcription comprises: 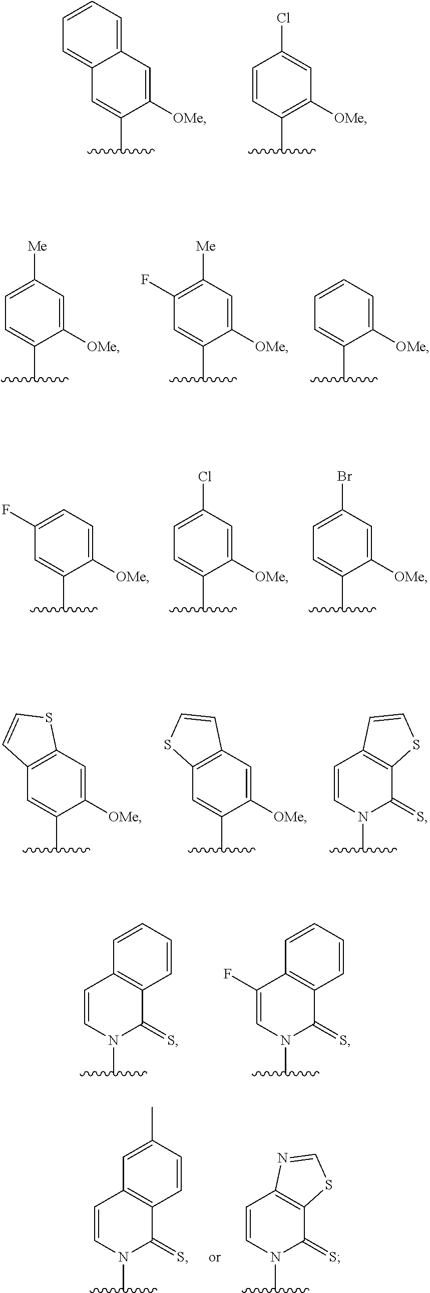
and/or
wherein at least one unnatural nucleotide that is incorporated into cDNA comprises: 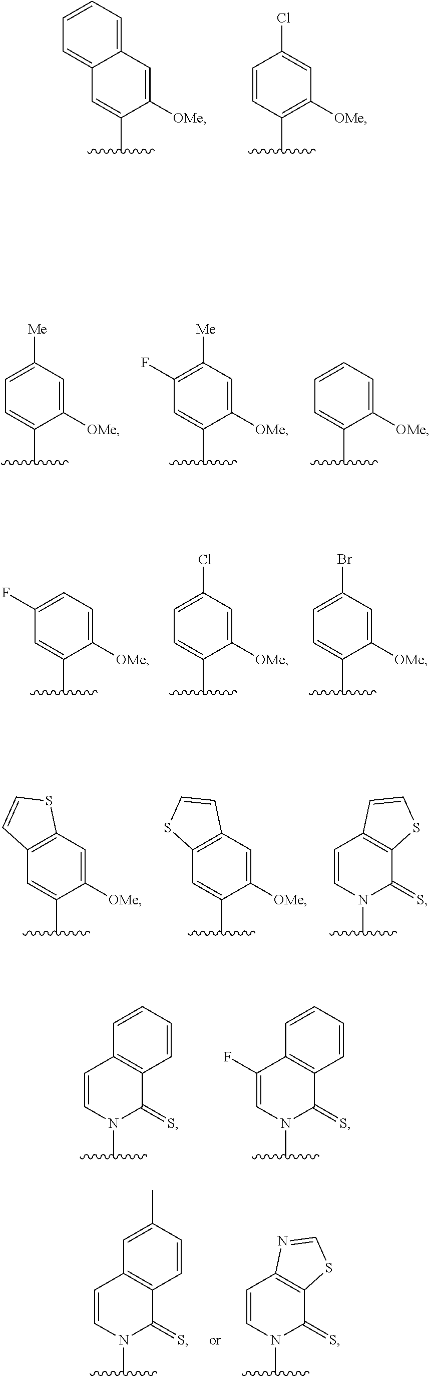
and optionally wherein the at least one unnatural nucleobase in the unnatural nucleotide is different from the at least one unnatural nucleobase in the polynucleotide that undergoes reverse transcription.

53. (canceled)

54. The method of claim 51, wherein the at least one unnatural nucleotide comprises: 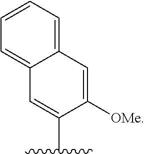 [SMILES: *c1cc2ccccc2cc1OC]
and/or wherein the at least one unnatural nucleotide comprises:

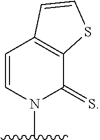 [SMILES: *n1ccc2ccsc2c1=S]

55. (canceled)

56. The method of claim 1, wherein the reverse transcriptase is Avian Myeloblastosis Virus (AMV) reverse transcriptase, Moloney Murine Leukemia Virus (MMLV) reverse transcriptase, Super Script II (SS II) reverse transcriptase, Super Script III (SS III) reverse transcriptase, Super Script IV (SS IV) reverse transcriptase, or Volcano 2G (V2G) reverse transcriptase; and/or
wherein the unnatural dNTP is not dTPT3TP, and/or
wherein the reverse transcribing takes place in vitro.

57. (canceled)

58. (canceled)

59. (canceled)


SEQUENCE LISTING (34 sequences)

SEQ ID NO: 1 - DNA, 49 bp, synthetic construct
  source 1..49: mol_type = other DNA
gacaaattaatacgactcactataggaaacctgatcatgtagatcgaac

SEQ ID NO: 2 - DNA, 21 bp, synthetic construct
  source 1..21: mol_type = other DNA
ccccaggctttacactttatg

SEQ ID NO: 3 - DNA, 39 bp, synthetic construct
  source 1..39: mol_type = other DNA
  modified_base 2: mod_base = OTHER; note = methylated g
tggcggaaaccccgggaatctaacccggctgaacggatt

SEQ ID NO: 4 - DNA, 20 bp, synthetic construct
  source 1..20: mol_type = other DNA
ggaatctaacccggctgaac

SEQ ID NO: 5 - DNA, 40 bp, synthetic construct
  source 1..40: mol_type = other DNA
ggaatctaacccggctgaaccctcgatgttgtggcggatc

SEQ ID NO: 6 - DNA, 44 bp, synthetic construct
  source 1..44: mol_type = other DNA
gattccattcttttgtttgtctgctggcggaaaccccgggaatc

SEQ ID NO: 7 - DNA, 44 bp, synthetic construct
  source 1..44: mol_type = other DNA
ggaatctaacccggctgaacgattccattcttttgtttgtctgc

SEQ ID NO: 8 - DNA, 40 bp, synthetic construct
  source 1..40: mol_type = other DNA
atgggtctcacacaaactcgagtacaactttaactcacac

SEQ ID NO: 9 - DNA, 33 bp, synthetic construct
  source 1..33: mol_type = other DNA
atgggtctcgattccattcttttgtttgtctgc

SEQ ID NO: 10 - DNA, 35 bp, synthetic construct
  source 1..35: mol_type = other DNA
atgggtctcgaaacctgatcatgtagatcgaacgg

SEQ ID NO: 11 - DNA, 28 bp, synthetic construct
  source 1..28: mol_type = other DNA
atgggtctcatctaacccggctgaacgg

SEQ ID NO: 12 - DNA, 19 bp, synthetic construct
  source 1..19: mol_type = other DNA
taatacgactcactatagg

SEQ ID NO: 13 - DNA, 63 bp, synthetic construct
  source 1..63: mol_type = other DNA
ctcgagtacaactttaactcacacaatgtatacatcacggcagacaaacaaaagaatggaatc

SEQ ID NO: 14 - DNA, 63 bp, synthetic construct
  source 1..63: mol_type = other DNA
ctcgagtacaactttaactcacacaatgtagtaatcacggcagacaaacaaaagaatggaatc

SEQ ID NO: 15 - DNA, 63 bp, synthetic construct
  modified_base 32: mod_base = OTHER; note = unnatural ribonucleotide NaM
  source 1..63: mol_type = other DNA
ctcgagtacaactttaactcacacaatgtaancatcacggcagacaaacaaaagaatggaatc

SEQ ID NO: 16 - DNA, 63 bp, synthetic construct; identical to SEQ ID NO: 15
  modified_base 32: mod_base = OTHER; note = unnatural ribonucleotide TPT3
  source 1..63: mol_type = other DNA

SEQ ID NO: 17 - DNA, 63 bp, synthetic construct
  modified_base 32: mod_base = OTHER; note = unnatural ribonucleotide NaM
  source 1..63: mol_type = other DNA
ctcgagtacaactttaactcacacaatgtagncatcacggcagacaaacaaaagaatggaatc

SEQ ID NO: 18 - DNA, 63 bp, synthetic construct; identical to SEQ ID NO: 17
  modified_base 32: mod_base = OTHER; note = unnatural ribonucleotide TPT3
  source 1..63: mol_type = other DNA

SEQ ID NO: 19 - DNA, 63 bp, synthetic construct
  modified_base 32: mod_base = OTHER; note = unnatural ribonucleotide NaM
  source 1..63: mol_type = other DNA
ctcgagtacaactttaactcacacaatgtagntatcacggcagacaaacaaaagaatggaatc

SEQ ID NO: 20 - DNA, 63 bp, synthetic construct; identical to SEQ ID NO: 19
  modified_base 32: mod_base = OTHER; note = unnatural ribonucleotide TPT3
  source 1..63: mol_type = other DNA

SEQ ID NO: 21 - DNA, 63 bp, synthetic construct
  modified_base 32: mod_base = OTHER; note = unnatural ribonucleotide NaM
  source 1..63: mol_type = other DNA
ctcgagtacaactttaactcacacaatgtaanaatcacggcagacaaacaaaagaatggaatc

SEQ ID NO: 22 - DNA, 63 bp, synthetic construct
  modified_base 32: mod_base = OTHER; note = unnatural ribonucleotide NaM
  source 1..63: mol_type = other DNA
ctcgagtacaactttaactcacacaatgtaantatcacggcagacaaacaaaagaatggaatc

SEQ ID NO: 23 - DNA, 63 bp, synthetic construct
  modified_base 32: mod_base = OTHER; note = unnatural ribonucleotide NaM
  source 1..63: mol_type = other DNA
ctcgagtacaactttaactcacacaatgtatnaatcacggcagacaaacaaaagaatggaatc

SEQ ID NO: 24 - DNA, 63 bp, synthetic construct
  modified_base 32: mod_base = OTHER; note = unnatural ribonucleotide NaM
  source 1..63: mol_type = other DNA
ctcgagtacaactttaactcacacaatgtatntatcacggcagacaaacaaaagaatggaatc

SEQ ID NO: 25 - DNA, 63 bp, synthetic construct
  modified_base 32: mod_base = OTHER; note = unnatural ribonucleotide NaM
  source 1..63: mol_type = other DNA
ctcgagtacaactttaactcacacaatgtagnaatcacggcagacaaacaaaagaatggaatc

SEQ ID NO: 26 - DNA, 52 bp, synthetic construct
  source 1..52: mol_type = other DNA
cctgatcatgtagatcgaacggactgtaaatccgttcagccgggttagattc

SEQ ID NO: 27 - DNA, 52 bp, synthetic construct
  source 1..52: mol_type = other DNA
cctgatcatgtagatcgaacggactctaaatccgttcagccgggttagattc

SEQ ID NO: 28 - DNA, 52 bp, synthetic construct
  modified_base 27: mod_base = OTHER; note = unnatural ribonucleotide TPT3
  source 1..52: mol_type = other DNA
cctgatcatgtagatcgaacggactgntaatccgttcagccgggttagattc

SEQ ID NO: 29 - DNA, 52 bp, synthetic construct; identical to SEQ ID NO: 28
  modified_base 27: mod_base = OTHER; note = unnatural ribonucleotide NaM
  source 1..52: mol_type = other DNA

SEQ ID NO: 30 - DNA, 52 bp, synthetic construct
  modified_base 27: mod_base = OTHER; note = unnatural ribonucleotide TPT3
  source 1..52: mol_type = other DNA
cctgatcatgtagatcgaacggactgncaatccgttcagccgggttagattc

SEQ ID NO: 31 - DNA, 52 bp, synthetic construct; identical to SEQ ID NO: 30
  modified_base 27: mod_base = OTHER; note = unnatural ribonucleotide NaM
  source 1..52: mol_type = other DNA

SEQ ID NO: 32 - DNA, 52 bp, synthetic construct
  modified_base 27: mod_base = OTHER; note = unnatural ribonucleotide TPT3
  source 1..52: mol_type = other DNA
cctgatcatgtagatcgaacggactancaatccgttcagccgggttagattc

SEQ ID NO: 33 - DNA, 52 bp, synthetic construct; identical to SEQ ID NO: 32
  modified_base 27: mod_base = OTHER; note = unnatural ribonucleotide NaM
  source 1..52: mol_type = other DNA

SEQ ID NO: 34 - DNA, 52 bp, synthetic construct
  modified_base 27: mod_base = OTHER; note = unnatural ribonucleotide TPT3
  source 1..52: mol_type = other DNA
cctgatcatgtagatcgaacggacttncaatccgttcagccgggttagattc